(19) United States
(12) Patent Application Publication
(10) Pub. No.: US 2021/0205775 A1
(43) Pub. Date: Jul. 8, 2021

(54) SOLID-PHASE POLYMER SYNTHESIS ON REUSABLE SUBSTRATES

(71) Applicant: MICROSOFT TECHNOLOGY LICENSING, LLC, Redmond, WA (US)

(72) Inventors: Bichlien NGUYEN, Seattle, WA (US); Yuan-Jyue CHEN, Seattle, WA (US); Jake SMITH, Seattle, WA (US)

(21) Appl. No.: 16/735,628

(22) Filed: Jan. 6, 2020

(51) Int. Cl. B01J 19/00; C07K 1/04

(52) U.S. Cl. CPC B01J 19/0046; C07K 1/04; B01J 2219/00605; B01J 2219/00722; B01J 2219/00725; B01J 2219/00596

(57) ABSTRACT

Substrates for solid-phase synthesis are reused by freeing synthesized polymers without removing the linkers that hold the polymers to the substrate. The linkers may be made of oligonucleotides or polypeptides. In an implementation, the polymers are released by cleavage of the linkers and then the truncated linkers are regenerated by adding back the portion that was removed. In an implementation, molecular bonds between the linkers and the polymers are cleaved releasing the polymers while leaving the linkers available for reuse without regeneration. In an implementation, single-stranded oligonucleotide linkers are hybridized to complementary strands that hold the polymers to the substrate with double-stranded oligonucleotide complexes. The double-stranded oligonucleotide complexes are denatured releasing the polymers while leaving the original linkers attached to the substrate. The polymers that are synthesized with these techniques may be the same or different type of molecules than the linkers.

### BACKGROUND

Solid-phase synthesis creates polymers by growing the polymer strands on a solid substrate in a step-by-step process. Solid-phase synthesis can be more efficient, faster, and simpler than synthesis in a liquid state. There are established techniques for solid-phase synthesis of common biological polymers such as oligonucleotides and polypeptides as well as for other polymers. The specific solid-phase synthesis technique, of course, differs according to the type of polymer. Oligonucleotides, for example, may be synthesized using the well-known phosphoramidite method in which phosphoramidite building blocks are sequentially coupled to a growing oligonucleotide chain in a particular sequence.

The solid substrate is used as a platform on which the polymers are grown. Common structures for substrates are flat surfaces such as “chips” and small beads. Linker molecules are sometimes used to anchor the polymers to the solid substrate during synthesis. Linkers are capable of forming bonds to both molecules on the surface of the substrate and to molecules in the polymers. After synthesis is complete, the polymers are released from the surface of the substrate by breaking the linkers typically through a chemical reaction that destroys the linkers. Once released from the surface of the substrate, the polymers may be collected and used. The substrates are then discarded or completely stripped and re-coated with new linkers.

However, continually creating new substrates, or repeatedly adding linkers to stripped substrates, can be wasteful and inefficient especially when performing polymer synthesis at scale. Techniques, substrates, and linkers for solid-phase synthesis that reduce waste and increase efficiency are particularly beneficial for applications that synthesize large amounts of polymers. One emerging application for large-scale deoxyribonucleic acid (DNA) synthesis is storage of digital data. In this technique for data storage, artificial DNA strands are synthesized in which the order of nucleotide bases encodes binary digits. The following disclosure is made with respect to these and other considerations.

### SUMMARY

This disclosure describes reusable substrates for solid-phase synthesis of polymers. Solid-phase synthesis is a method in which molecules are bound on a solid substrate and synthesized step-by-step in a single reaction vessel. Benefits compared with normal synthesis in a liquid state include higher efficiency and throughput as well as increased simplicity and speed. The techniques of this disclosure may be used with any type of solid-phase synthesis including oligonucleotide synthesis and polypeptide synthesis. The surface of the solid substrate may be coated with molecules that function as handles or linkers binding the polymers to the substrate during synthesis.

The linkers themselves may also be, but are not necessarily, polymers such as oligonucleotides or polypeptides. The substrates provided in this disclosure are reusable because the linkers remain attached to the substrate after the polymers are released from the substrate. Thus, after one round of synthesis is complete, the substrate can be used again for another round of synthesis. There may be cleaning or washing steps between each round of synthesis. Reusing the same substrate and linkers multiple times reduces waste and improves efficiencies compared to techniques that discard the substrate after a single round of synthesis or techniques that completely strip the surface of the substrate and reattach entirely new linkers.

In an implementation, the linkers are cleaved to release the polymers from the substrate. Oligonucleotide linkers may be cleaved, for example, by an endonuclease or Crispr-Cas9 system. Polypeptide linkers may be cleaved, for example, by a protease or chemical cleavage agent. Cleavage within a linker leaves a portion of the linker attached to the substrate and a portion attached to the polymer. The truncated portion of the linker that remains attached to the substrate is regenerated to its original full length prior to reuse. For oligonucleotide linkers, template strands and polymerase may be used to extend truncated linkers. Alternatively, pre-synthesized oligonucleotide fragments that replace the portion cleaved off may be ligated onto the ends of the truncated linkers. For polypeptide linkers, conventional solid-phase synthesis of peptides (SPPS) may be used to extend the truncated linker.

In an implementation, synthesized polymers are cleaved from the linkers at the point of connection to the linkers. Generally, a covalent bond between the end of the linker and the first monomer in the synthesized polymer is broken. Because the linker is not truncated in this technique, it remains at its original full length and may be reused in a later round of synthesis. An endonuclease with a cut site that is separate from its recognition site may be used to cleave at the end, rather than in the middle, of an oligonucleotide linker. An oligonucleotide linker that is a DNA-ribonucleic acid (RNA) hybrid may be designed so that inclusion of ribonucleotides causes an enzyme to cleave the nucleotide at the end of the linker. Proteases that cut at the end of a recognition sequence may be used to cleave at the end rather than the middle of a polypeptide linker. Polypeptide linkers used for synthesis of oligonucleotides may be cleaved at the point of connection between the polypeptide linker and the oligonucleotide strand by an enzyme that recognizes the peptide-nucleotide bond such as the 23S ribosomal RNA from Thermus aquaticus.

In an implementation, oligonucleotide linkers may use the hybridization of complementary single-stranded oligonucleotides to hold the polymers (which are also oligonucleotides in this implementation) to the surface of the substrate. The linker is a single-stranded oligonucleotide without a covalent attachment to the polymer. A second single-stranded oligonucleotide, an adaptor strand, hybridizes to the linker. The adaptor strand is either extended to become part of the polymer itself or holds the polymer to the linker by hybridizing to both the linker and the polymer. Heat or another technique is used to denature the double-stranded oligonucleotide structure formed from the linker and the adaptor strand. This releases the polymer while the original linker remains attached to the substrate.

This Summary is provided to introduce a selection of concepts in a simplified form that are further described below in the Detailed Description. This Summary is not intended to identify key features or essential features of the claimed subject matter nor is it intended to be used to limit the scope of the claimed subject matter. The term “techniques,” for instance, may refer to system(s) and/or method(s) as permitted by the context described above and throughout the document.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items. Structures shown in the figures are representative and not necessarily to scale.

FIG. 1 shows examples of linkers attached to a substrate.

FIG. 2 shows cleavage and regeneration of a polypeptide linker.

FIG. 3 shows cleavage of a single-stranded oligonucleotide linker.

FIG. 4 shows regeneration of a truncated single-stranded oligonucleotide linker using polymerase and nucleotides.

FIG. 5 shows regeneration of a truncated single-stranded oligonucleotide linker using a linker replacement strand and ligase.

FIG. 6 is a flow diagram showing an illustrative process for solid-phase polymer synthesis that includes reusing a substrate by regenerating cleaved linkers.

FIG. 7 is a flow diagram showing an illustrative process for solid-phase polymer synthesis that includes regenerating single-stranded oligonucleotide linkers using polymerase and nucleotides to extend truncated oligonucleotide linkers.

FIG. 8 is a flow diagram showing an illustrative process for solid-phase polymer synthesis that includes regenerating single-stranded oligonucleotide linkers by ligating a linker replacement strand to the end of truncated oligonucleotide linkers.

FIG. 9 shows cleavage at the point of attachment between a linker and a polymer strand.

FIG. 10 is a flow diagram showing an illustrative process for solid-phase polymer synthesis that includes separating polymer strands from linkers by cleaving at the point of attachment between a linker and a polymer strand.

FIG. 11 shows use of a single-stranded oligonucleotide linker with its 3′-end attached to the surface of a substrate as the linker for solid-phase synthesis of an oligonucleotide.

FIG. 12 shows use of a single-stranded oligonucleotide linker with its 5′-end attached to the surface of a substrate as the linker for solid-phase synthesis of an oligonucleotide.

FIG. 13 is a flow diagram showing an illustrative process for solid-phase polymer synthesis using single-stranded oligonucleotide linkers.

FIG. 14 shows a solid substrate patterned with multiple types of linkers.

### DETAILED DESCRIPTION

There many types of linkers that can be used to attach polymer strands to a substrate during solid-phase synthesis. Many are artificial molecules that are cleaved chemically and irreversibly to release the polymer strands. Non-covalent attachment such as streptavidin-biotin interactions may also be used to attach polymer strands to a solid substrate. Some examples of linkers are provided in U.S. patent application Ser. No. 16/230,787 filed on Dec. 21, 2018, with the title “Selectively Controllable Cleavable Linkers.” However, most of these linkers are not suitable for reuse.

Certain biological polymers such as oligonucleotides and polypeptides can be adapted for use as reusable linkers. Oligonucleotides and polypeptides have properties that other chemical linkers do not: enzymes exist that can cleave at specific locations and it is possible to synthesize oligonucleotides and polypeptides in situ while attached to a substrate. Additionally, the ability of single-stranded oligonucleotides to hybridize with complementary strands provides opportunities for creating reusable linkers from single-stranded oligonucleotides. All of the linkers presented in this disclosure bind polymer strands to a solid substrate during synthesis and then release the synthesized polymer strands in ways that leave the linkers available for reuse with little or no processing. Additionally, the techniques for cleaving the linkers, or otherwise releasing the polymers, may avoid the use of harsh chemicals that could damage the surface of a solid substrate.

The solid substrates discussed in this disclosure may be made of many types of materials including, but not limited to, glass, silicon, metal, and plastic. Depending on the material of the solid substrate, it may be functionalized to provide locations for chemical attachment of the linkers. Once functionalized, if necessary, linkers are attached to the surface of the solid substrate.

In various implementations, the linkers may be oligonucleotides or polypeptides. Oligonucleotides include both DNA, RNA, and hybrids containing mixtures of DNA and RNA. DNA includes nucleotides with one combination of the four natural bases cytosine (C), guanine (G), adenine (A), or thymine (T) as well as unnatural bases, noncanonical bases, and modified bases. RNA includes nucleotides with one of the four natural bases cytosine, guanine, adenine, or uracil (U) as well as unnatural bases, noncanonical bases, and modified bases. Polypeptides have multiple amino acids linked via amide bonds, also known as peptide bonds. Amino acid as used herein includes all 20 standard amino acids, all 22 natural amino acids, non-proteinogenic amino acids, and D-isomers.

Once patterned with linkers, solid-phase synthesis can be performed by growing polymer strands from the ends of the attached linkers. The linkers and techniques described herein are also compatible with many different types of solid-phase polymer synthesis such as oligonucleotide synthesis and polypeptides synthesis. Thus, in some implementations, the linkers may be the same type of polymer as the polymers being synthesized. For example, DNA linkers may be used to grow DNA strands. Polypeptide linkers may be used to grow protein strands.

Oligonucleotide synthesis can be performed by phosphoramidite-based methods or by enzymatic methods. Phosphoramidite-based synthesis uses a cycle of four different monomer mixtures to add each individual nucleoside. Addition is typically performed in a 3′ to 5′ synthesis direction but synthesis in the 5′ to 3′ direction is also possible although it is less efficient. Phosphoramidite synthesis is performed in a solution of an organic solvent such as acetonitrile. Enzymatic synthesis of DNA uses a template-independent DNA polymerase, terminal deoxynucleotidyl transferase (TdT), which is an enzyme that evolved to rapidly catalyze the linkage of naturally occurring nucleosides in a 5′ to 3′ direction. TdT adds nucleotides indiscriminately so it is stopped from continuing unregulated synthesis by various techniques such a tethering the TdT, creating variant enzymes, and using nucleotides that include reversible terminators to prevent chain elongation. Enzymatic synthesis is performed in an aqueous solution.

Solid-phase synthesis of peptides (SPPS) is a technique for synthesizing polymer strands that may be used with the linkers and techniques provided in this disclosure. SPPS allows the rapid assembly of a peptide chain through successive reactions of amino acid derivatives on an insoluble porous substrate. The general SPPS procedure is one of repeated cycles of alternate N-terminal deprotection and coupling reactions. The cycles are repeated until the desired sequence has been synthesized. SPPS cycles may also include capping steps that block the ends of unreacted amino acids from reacting.

Unless otherwise specified, hybridization, as used throughout this disclosure, refers to the capacity for hybridization between two single-stranded oligonucleotides or oligonucleotide segments at 37° C. in 1× TAE buffer containing 40 mM TRIS base, 20 mM acetic acid, 1 mM ethylenediaminetetraacetic acid (EDTA), and 12.5 mM MgCl₂. As is known to those of ordinary skill in the art, conditions of temperature and ionic strength determine the “stringency” of the hybridization. Suitable conditions for oligonucleotide hybridization and washing conditions are provided in many reference manuals such as, for example, Michael R. Green & Joseph Sambrook, Molecular Cloning: A Laboratory Manual, Cold Spring Harbor Laboratory Press, 4^{th} ed. (2012).

It is understood the sequence of an oligonucleotide need not be 100% complementary to that of its target to be specifically hybridizable. Moreover, the oligonucleotide may hybridize over one or more segments such that intervening or adjacent segments are not involved in the hybridization event (e.g., a loop structure or hairpin structure). The oligonucleotide can comprise at least 20%, at least 30%, at least 40%, at least 50%, at least 60%, at least 70%, at least 80%, at least 90%, at least 95%, at least 99%, or 100% sequence complementarity to a target region within the target oligonucleotide sequence to which they are targeted. The degree to which two oligonucleotides are complementary may also be defined in terms of the number of complementary base pairs. For example, oligonucleotides may be hybridizable if they have at least 5, at least 10, at least 15, at least 20, or more complementary base pairs.

For example, a complementary oligonucleotide in which 18 of 20 base pairs of the oligonucleotide are complementary to a target region, and would therefore specifically hybridize, represents 90% complementarity. In this example, the remaining non-complementary nucleotides may be clustered or interspersed with complementary nucleotides and need not be contiguous to each other or to complementary nucleotides. As a further example, two oligonucleotides each with 100 nucleotides may hybridize if they share a region in which 20 base pairs are complementary. Percent complementarily between particular stretches of oligonucleotide sequences can be determined routinely using software such as the BLAST programs (basic local alignment search tools) and PowerBLAST programs known in the art (Altschul et al., J. Mol. Biol., 1990, 215, 403-410; Zhang & Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

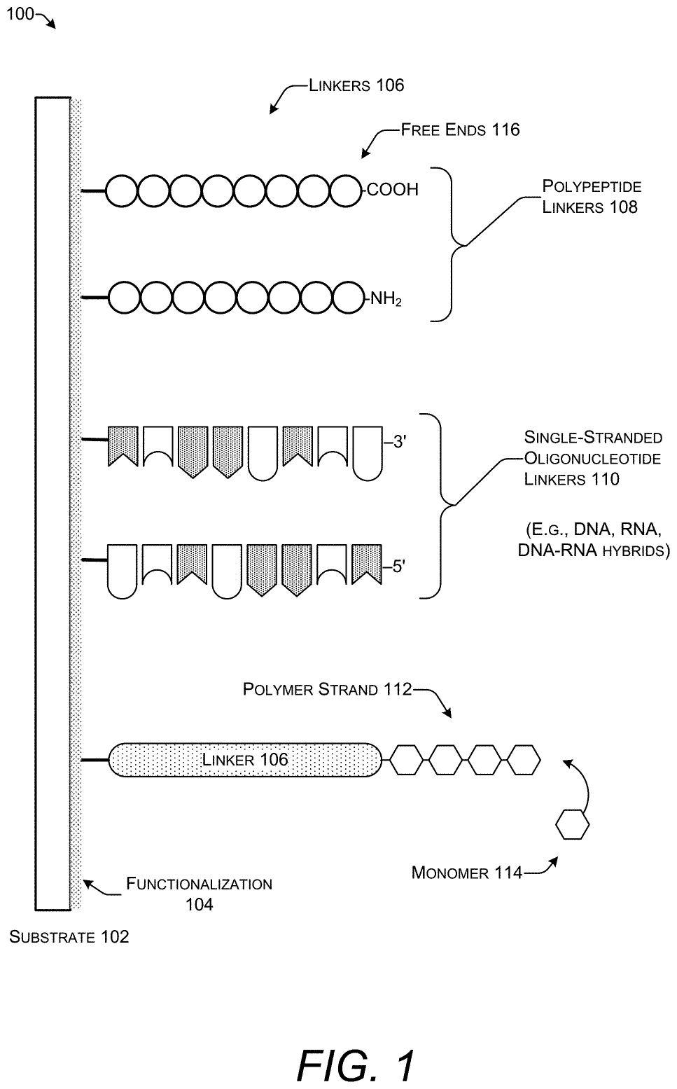

FIG. 1 is a schematic 100 showing a substrate 102 that may include functionalization 104 and multiple types of linkers 106. The linkers 106 may be polypeptide linkers 108 or single-stranded oligonucleotide linkers 110. The linkers 106, of either type, may be used in solid-phase polymer synthesis to grow a polymer strand 112 by sequential polymerization of monomers 114. The polymer strand 112 may be an oligonucleotide strand or a polypeptide strand. Thus, the monomers 114 may be nucleotides or amino acids. Polymer synthesis may proceed according to conventional solid-phase synthesis techniques appropriate for the type of polymer being synthesized. The polymer strand 112 may, in some implementations, be created by oligonucleotide assembly techniques that hybridize multiple pre-synthesized oligonucleotide units together to create an oligonucleotide chain with a specific sequence. Examples of suitable oligonucleotide assembly techniques that may be used with the contents of this disclosure are provided in U.S. patent application Ser. No. 16/698,860 filed on Nov. 27, 2019, with the title “Oligonucleotide Assembly Using Electrically Controlled Hybridization.”

The linkers 106 may be polypeptide linkers 108. Polypeptide linkers 108 are peptide chains of any length. In some implementations, each polypeptide linker 108 includes about 5-10 amino acids which may have specific, pre-determined peptide sequence. The polypeptide linkers 108 may be created from amino acids primarily with non-reactive side groups to minimize the formation of branched structures. For example, amino acids used in the peptide linkers 108 may be selected from the group of nonpolar, aliphatic amino acids that includes alanine, valine, leucine, methionine, proline, and isoleucine. More specifically, the amino acids used in the peptide linkers 108 may be one of glycine, alanine, or valine. In one implementation, the polypeptide linkers 108 may be glycine chains that include 5-8 amino acids.

However, the polypeptide linkers 108 may include amino acids with reactive side groups that are recognized by proteases which cleave the polypeptide linkers 108. To minimize the possibility of off-target cleavage, amino acids in a polypeptide linker 108 other than at an enzyme recognition site may be selected from the group of nonpolar, aliphatic amino acids. For example, amino acids positioned between the substrate 102 and a cleavage site may be selected from the group of nonpolar, aliphatic amino acids and amino acids in the cleavage site may include reactive side groups.

The polypeptide linkers 108 may be oriented with respect to the substrate 102 so that either the amino terminal (N-terminus) or the carboxyl terminal (C-terminus) is attached to the substrate 102. The end of a polypeptide linker 108 that is not attached to the substrate 102, the free end 116, connects to the polymer strand 112. If the polymer strand 112 is itself also a polypeptide, the connection is a peptide bond. If the polymer strand 112 is an oligonucleotide, the connection may be made by established techniques for creating peptide-oligonucleotide conjugates (POCs). Peptide-oligonucleotide conjugates (POCs) are covalent constructs that link oligonucleotides to synthetic peptide sequences. See Yashveer Singh et al., Chemical Strategies for Oligonucleatide-Conjugates Synthesis. 12 Curr. Org. Chem. 263 (2008).

The linkers 106 may be single-stranded oligonucleotide linkers 110. The single-stranded oligonucleotide linkers 110 are single-stranded DNA, RNA, or hybrid DNA-RNA molecules. In some implementations, the oligonucleotide linkers 110 are about 10-20 base pairs long. The oligonucleotide linkers 110 may be created with specific, pre-determined sequences of nucleotides. The single-stranded oligonucleotide linkers 110 may be oriented with respect to the substrate 102 so that either the 3′-end or the 5′-end is attached to the substrate 102. The end of the single-stranded oligonucleotide linker 110 that is not attached to the substrate 102, the free end 116, connects to the polymer strand through a reactive group such as the 3′-hydroxyl group, 2′-hydroxyl group in RNA, the 5′-phosphate, or a group on the base of the final nucleotide. If the polymer strands 112 are oligonucleotides, the connection is a phosphodiester bond. If the polymer strands 112 are polypeptides, the connection may be made by established techniques for creating POCs.

The substrate 102 is a solid object having one or more rigid surfaces. The substrate 102 can have any size and shape such as generally spherical beads or a generally fiat “chip.” The substrate 102 may be part of a larger object formed from the same or different material. The solid substrate 102 can be comprised of any of glass, a silicon material, a metal material, plastic, or combination thereof. Examples of suitable substrates 102 that may be used with the contents of this disclosure are provided in U.S. patent application Ser. No. 16/597,799 filed on Oct. 9, 2019, with the title “High Surface Area Coatings for Solid-Phase Synthesis.”

In some implementations, the substrate 102 is formed from glass. As used herein, glass is a non-crystalline, amorphous solid. Glass may be soda lime glass or borosilicate glass which are both commonly used in glass slides. One preparation of glass that may be used for solid-phase synthesis is porous glass such as controlled pore glass (CPG). CPG glass may be pre-derivatized to contain DNA synthesis linker functional groups (e.g., silane groups) on its surface. It may be used either as a bulk packed-bed column or embedded in a sintered plastic frit for traditional large-scale oligonucleotide synthesis. The column or frit provides a structure to prevent migration of the small glass particles or beads.

Silicon materials comprise silicon (Si), silicon oxides, silicon nitrides, and combinations thereof. Silicon refers to solid, metallic silicon. Silicon oxides include silicon dioxide (SiO₂). Silicon nitrides include chemical compounds of the elements silicon and nitrogen. The most thermodynamically stable silicon nitride is Si₃N₄. Unless otherwise specified, as used herein silicon nitride refers to Si₃N₄.

Metal materials comprise metals, metal oxides, metal nitrides, and combinations thereof such as alloys of two or more metals. As used herein, metal materials do not include silicon materials. In some implementations, the metal is a period 4 transition metal. The period 4 transition metals are scandium (Sc), titanium (Ti), vanadium (V), chromium (Cr), manganese (Mn), iron (Fe), cobalt (Co), nickel (Ni), copper (Cu), and zinc (Zn). In some implementations, the metal is a metal from the boron group. The boron group of metals includes aluminum (Al), gallium (Ga), indium (In), and thallium (Ti). In some implementations, the metal is a noble metal. Noble metals are gold (Au), platinum (Pt), silver (Ag), and palladium (Pd). In some implementations, the metal is stainless steel. Stainless steel is a steel alloy with a minimum of about 10.5% chromium content by mass and a maximum of about 1.2% carbon by mass. Metal oxides include oxides of any of the metals listed above. Metal nitrides include nitrides of any of the metals listed above.

Plastics include thermoplastics, polymeric organosilicon compounds, synthetic rubber, polyimide, and combinations thereof. Thermoplastics include polycarbonates, polypropylene, polyethylene, and polyoxymethylene (POM). Polypropylene is a thermoplastic polymer that belongs to the group of polyolefins. Polyethylene is a class of polymers of which most, but not all, have the chemical formula (C₂H₄)ₙ. Polymeric organosilicon compounds include polydimethylsiloxane (PDMS), also known as dimethylpolysiloxane or dimethicone. Synthetic rubber includes ethylene propylene diene monomer (EPDM) rubber and Viton®. There are multiple compositions of Viton® all of which are mixtures of one or more of copolymers of hexafluoropropylene (HFP) and vinylidene fluoride (VDF or VF2), terpolymers of tetrafluoroethylene (TFE), vinylidene fluoride (VDF), hexafluoropropylene (HFP), and perfluoromethylvinylether (PMVE). Polyimides include Kapton® which has the chemical formula poly (4,4′-oxydiphenylene-pyromellitimide).

The functionalization 104 may add functional groups include hydroxyl groups, amine groups, thiolate groups, alkenes, n-alkenes, alkalines, N-Hydroxysuccinimide (NHS)-activated esters, polyaniline, aminosilane groups, silanized oxides, oligothiophenes, and diazonium compounds. A hydroxyl group is a moiety with the formula OH. It contains oxygen bonded to hydrogen. Hydroxyl groups are present on some polysaccharides such as agarose. An amine group is an organic hydrocarbon that contains a basic nitrogen atom with a lone pair of electrons. Amines are derivatives of ammonia in which one or more hydrogen atoms are replaced by a substituent such as an alkyl or aryl group (i.e., alkylamines and arylamines; amines in which both types of substituent are attached to one nitrogen atom are called alkylarylamines). Amines can be classified according to the nature and number of substituents on nitrogen. Aliphatic amines contain only hydrogen and alkyl substituents. Aromatic amines have the nitrogen atom connected to an aromatic ring.

A thiolate group is any organosulfur compound of the form R—SH, where R represents an alkyl or other organic substituent. An alkene is an unsaturated hydrocarbon that contains at least one carbon-carbon double bond. A n-alkene is an unsaturated linear hydrocarbon. An alkyne is an unsaturated hydrocarbon containing at least one carbon-carbon triple bond.

An NHS-activated ester is reactive species that couples with amines efficiently. A silanized oxide is an oxide of silicon that forms a covalent —Si—O—Si— bonds. Polyaniline (PANI) is a conducting polymer of the semi-flexible rod polymer family. An oligothiophene is oligomer of a thiophene. Diazonium compounds are a group of organic compounds with the structure R—N⁺₂X⁻ where R can be any organic group, such as an alkyl or an aryl (e.g., aryl diazonium), and X is an inorganic or organic anion, such as a halogen.

### Cleavage of Linkers

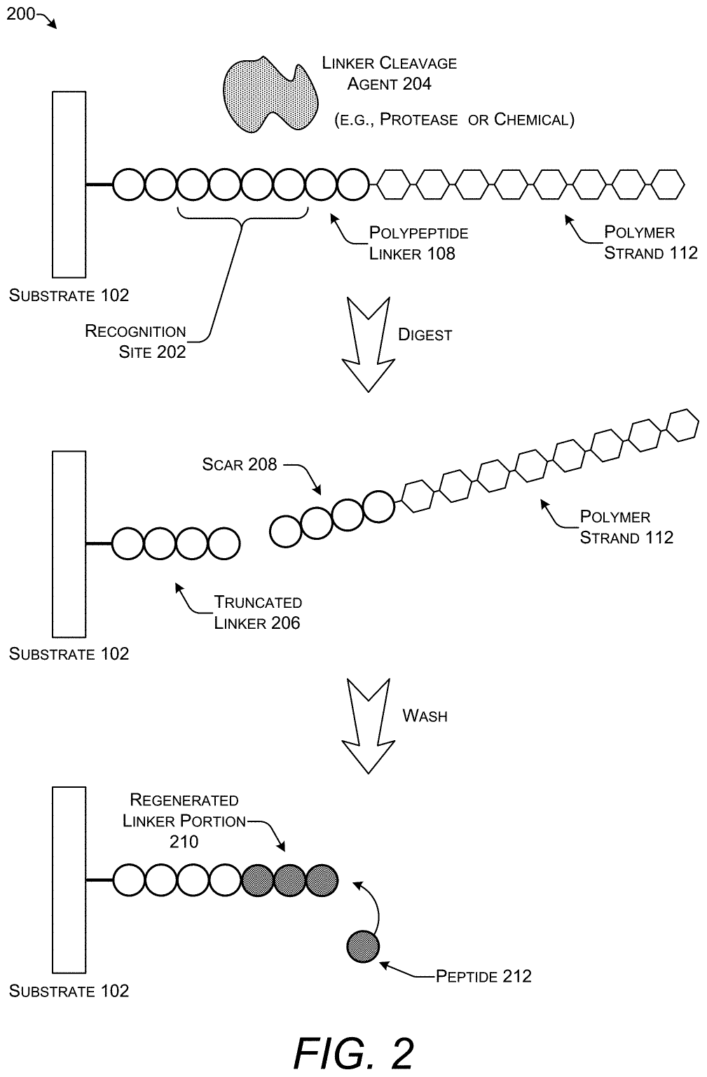

FIG. 2 shows an example time series 200 of cleaving and then regenerating a polypeptide linker 108. The polypeptide linker 108 may be designed to include a recognition site 202 that is cleaved by a linker cleavage agent 204. The linker cleavage agent 204 may be a protease that cleaves polypeptide chains or a non-enzymatic, chemical cleavage agent.

A protease (also called a peptidase or proteinase) is an enzyme that catalyzes proteolysis, the breakdown of proteins into smaller polypeptides or single amino acids by cleaving the peptide bonds within proteins by hydrolysis. Proteases that are highly specific and cleave only certain peptide sequences may be used to control where cleavage occurs. The polypeptide linker 108 is thus designed so that the recognition site 202 is recognized and cleaved by the protease. The linker cleavage agent 204 may be any one of a number of different proteases. One class of proteases are the endoproteinases. Endopeptidases or endoproteinases are proteolytic peptidases that break peptide bonds of nonterminal amino acids (i.e. within the molecule). Techniques for digesting a polypeptide with a protease may be obtained from the supplier of the protease and are well-known to those having ordinary skill in the art. Some suitable proteases are described below. Recognition sites are shown for the proteases in parentheses where X represents any peptide, specific peptides are represented by the standard International Union of Pure and Applied Chemistry (IUPAC) three-letter codes, and a downward arrow “↓” indicates where cleavage occurs.

Trypsin cleaves peptides on the C-terminal side of lysine and arginine residues. The rate of hydrolysis of this reaction is slowed if an acidic residue is on either side of the cleavage site and hydrolysis is stopped if a proline residue is on the carboxyl side of the cleavage site. Chymotrypsin is a serine protease that hydrolyzes peptide bonds with aromatic or large hydrophobic side chains (Tyr, Trp, Phe, Met, Leu) on the carboxyl end of the bond.

Thermolysin is a thermostable (thermophilic) extracellular metalloendopeptidase containing four calcium ions. Cofactors are zinc and calcium. Hydrolyzes protein bonds on the N-terminal side of hydrophobic amino acid residues. Thermolysin has a low cleavage specificity. Preferential cleavage: X↓Y-Z where X=any amino acid; Y=Leu, Phe, Ile, Val, Met, Ala, and Z is any amino acid other than Pro. Cleavage N-terminal to Leu is preferred over cleavage of N-terminal to Phe which is preferred over the others.

Pepsin hydrolyzes only peptide bonds, not amide or ester linkages. The cleavage specificity includes peptides with an aromatic acid on either side of the peptide bond, especially if the other residue is also an aromatic or a dicarboxylic amino acid. Increased susceptibility to hydrolysis occurs if there is a sulfur-containing amino acid close to the peptide bond, which has an aromatic amino acid. Pepsin will also preferentially cleave at the carboxyl side of phenylalanine and leucine, and to a lesser extent at the carboxyl side of glutamic acid residues. It does not cleave at valine, alanine, or glycine linkages.

Arg-C is a serine endoprotease that hydrolyzes peptide bonds at the carboxyl side of arginyl residues. Arg-C has been shown to cleave Lys-Lys and Lys-Arg bonds, and all Arg-X bonds may not be hydrolyzed. AspN (flavastacin) is a zinc metalloendopeptidase that selectively cleaves protein and peptide bonds N-terminal to aspartic acid residues (XX↓Asp-XXX). GluC is a serine proteinase that preferentially cleaves peptide bonds C-terminal to glutamic acid residues (XX-Glu↓XXX). It also cleaves at aspartic acid residues at a rate 100-300 times slower than at glutamic acid residues. LysC is a serine endoproteinase that cleaves peptide bonds at the carboxyl side of lysine (XX-Lys↓XXX).

TEV Protease, also known as Tobacco Etch Virus (TEV) Protease, is a highly specific cysteine protease that recognizes the amino-acid sequence Glu-Asn-Leu-Tyr-Phe-Gln-(Gly/Ser) (SEQ ID NO: 1) and cleaves between the Gln and Gly/Ser residues (i.e., Glu-Asn-Leu-Tyr-Phe-Gln↓(Gly/Ser)).

Enterokinase is a specific protease that cleaves after lysine at its cleavage site Asp-Asp-Asp-Asp-Lys (SEQ ID NO: 2) (i.e., Asp-Asp-Asp-Asp-Lys↓). It will sometimes cleave at other basic residues, depending on the conformation of the protein substrate. Clostripain is a proteolytic enzyme with highly restricted substrate specificity for Arg-X peptide bonds (Arg↓X).

Granzyme B is a neutral serine protease that cleaves Asp residues. Proteinase K is a subtilisin-related serine protease that will hydrolyze a variety of peptide bonds. Glutamyl endopeptidase I is a family of extracellular bacterial serine proteases. Proteases of this group hydrolyze peptide bonds after the negatively charged glutamic acid or aspartic acid, with a higher preference for the former. Proline-endopeptidase is a proteolytic peptidase that is specific for proline (Pro). Thrombin is a Na+ activated allosteric serine protease that hydrolyzes peptide and ester bonds specifically at the carboxylic side of Arg. Staphylococcal peptidase I is a serine proteinase that preferentially cleaves between Glu-X and Asp-X peptide bonds.

Chemical cleavage agents may be used as a linker cleavage agent 204 instead of enzymes. Some reagents suitable for cleaving polypeptide linkers 108 include BNPS-skatole, cyanogen bromide (CBrN), formic acid, hydroxylamine, 2-iodosobenzoic acid (IBX), and 2-nitro-5-thiocyanatobenzoic acid. Techniques for using these reagents to digest polypeptides are known to those of ordinary skill in the art.

BNPS-Skatole is used to cleave the tryptophanyl peptide bond found in tryptophan-containing proteins. Specifically, cleavage occurs at peptide bonds after amino acids with available Cγ-Cδ double bonds such as tryptophan, tyrosine, and histidine. CBrN is an inorganic compound with the formula (CN)PR or BrCN. It cleaves peptides at the C-terminus of methionine. 2-nitro-5-thiocyanatobenzoic acid (NTCB) is used to cyanylate and cleave proteins at cysteine residues.

If the polymer strand 112 is itself also a polynucleotide, there is a possibility that the linker cleavage agent 204 could cleave the polymer strand 112 instead of the polypeptide linker 108. To prevent this, the sequence of the polypeptide linker 108 may be designed so that the recognition site 202 is not found within the polymer strand 112.

After cleavage of the polypeptide linker 108, the polymer strand 112 may be removed from the surface of the substrate 102 by washing or eluting using any suitable wash or elution buffer. A truncated linker 206 remains attached to the substrate 102. The length of the truncated linker 206 depends on the location of cleavage. The portion of the polypeptide linker 108 that remains attached to the polymer strand 112 is referred to as a scar 208. It may be desirable to cleave the polypeptide linker 108 as close to the polymer strand 112 as possible to minimize the length of the scar 208. Doing so also minimizes the number of peptides that need to be regenerated in order to re-create the full-length polypeptide linker 108.

The scar 208 remains attached to the polymer strand 112 yet it is not part of the sequence of the polymer strand 112. This scar 208 may be removed by techniques that selectively digest proteins if the polymer strand 112 is itself not a polypeptide. If the polymer strand 112 is an oligonucleotide, polymerase chain reaction (PCR) may be used to increase the number of copies of the polymer strand 112 thereby diluting and effectively removing those polymer strands 112 that are still attached to the scar 208.

The polypeptide linker 108 may be regenerated from the truncated linker 206 by addition of a regenerated linker portion 210 that includes the peptides 212 cleaved off by the linker cleavage agent 204. Conventional SPPS or other peptide synthesis techniques may be used to add peptides 212 to the free end of the truncated linker 206. This regenerates a peptide strand with the same sequence as the original polypeptide linker 108.

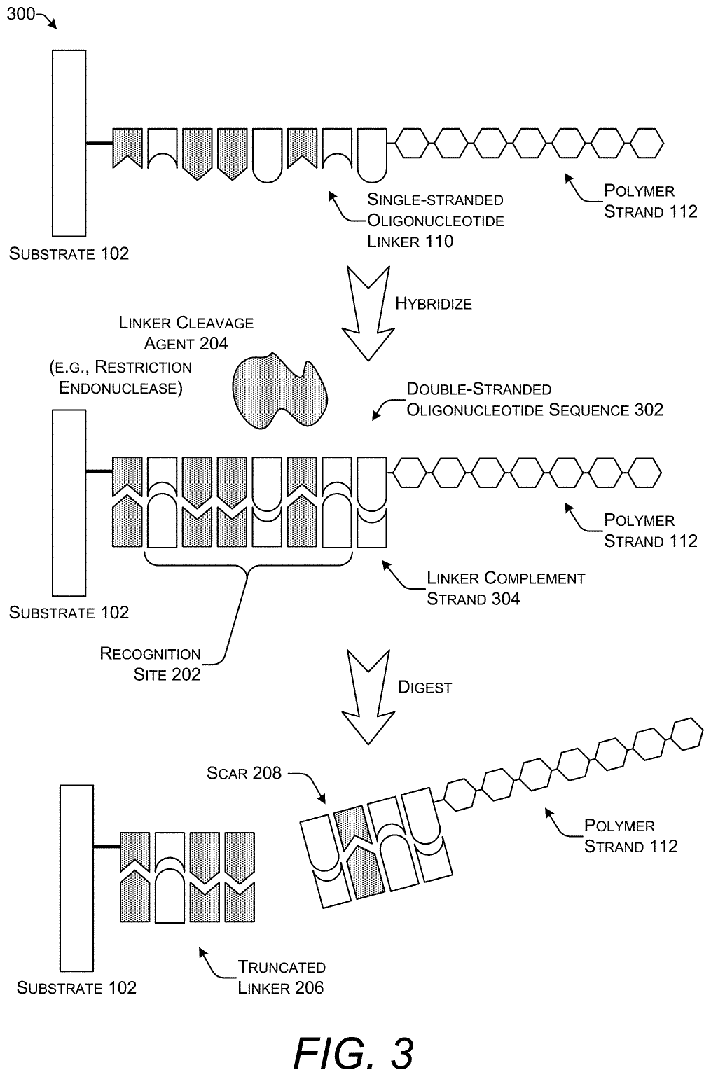

FIG. 3 shows an example time series 300 of cleaving a single-stranded oligonucleotide linker 110. A single-stranded oligonucleotide linker 110 as illustrated in FIG. 3 may be oriented with either its 5′-end or its 3′-end attached to the substrate 102. The sequence of the nucleotides in the single-stranded oligonucleotides linker 110 may be designed so that it includes a recognition site that is recognized by a linker cleavage agent 204 such as a restriction endonuclease. Restriction endonucleases are enzymes that cleave the phosphodiester bond within a polynucleotide chain at or near specific recognition sites known as restriction sites.

In some implementations, the linker cleavage agent 204 may recognize and cleave single-stranded oligonucleotides. One example of a linker cleavage agent 204 that performs site-specific cleavage of single-stranded DNA is the class-IIN restriction endonuclease, XcmI. See Shaw Pang-Chui & Mok Yu-Keung, XcmI as a universal restriction enzyme for single-stranded DNA, 133(1) Gene 85 (1993).

However, many endonucleases require a double-stranded sequence for recognition. In order to create a double-stranded oligonucleotide sequence 302 that includes the recognition site 202, a linker complement strand 304 may be added. The linker complement strand 304 is a single-stranded oligonudeotide that is complementary to all or a portion of the single-stranded oligonucleotide linker 110. The linker complement strand 304 may, at a minimum, be complementary to the recognition site 202.

The recognition site 202 in the double-stranded oligonucleotide sequence 302 may be cleaved by a linker cleavage agent 204 such as an endonuclease. Suitable endonucleases include type II restriction enzymes that recognize palindromic sequences of about 4-8 nucleotides in length and cleave within the recognition site 202 and type V restriction enzymes that use guide RNA's to target specific non-palindromic sequences (e.g. the CRISPR-Cas9 system). Techniques for performing restriction enzyme digests are available from the supplier of an endonuclease and are known to those of ordinary skill in the art. If the polymer strand 112 is also an oligonucleotide, then the recognition site 202 must be selected so that the same nucleotide sequence is not found within the polymer strand 112 in order to prevent cleavage of the polymer strand 112.

Cleavage of the single-stranded oligonucleotide linker 110 (either in its single-stranded form or as a double-stranded oligonucleotide sequence 302) releases the polymer strand 112 from the substrate 102 and creates a truncated linker 206. The truncated linker 206 remains attached to the substrate 102. A scar 208 may remain on the polymer strand 112. The scar 208 is a short sequence of nucleotides. The length of the scar 208 depends on the location of cleavage within the single-stranded oligonucleotide linker 110. It may be desirable to cleave the single-stranded oligonucleotide linker 110 as close to the polymer strand 112 as possible in order to minimize the length of the scar 208. Doing so also minimizes the number of nucleotides that need to be regenerated in order to re-create the full-length single-stranded oligonucleotide linker 110.

If the polymer strand 112 is not itself an oligonucleotide strand, the scar 208 may be removed by a step that digests oligonucleotides such as by addition of an exonuclease. If the polymer strand 112 is also an oligonucleotide strand, PCR amplification may be used to increase the concentration of the polymer strand 112 and effectively remove the polymer strands 112 that have scars 208 by dilution.

If a linker complement strand 304 was used to create a double-stranded oligonucleotide sequence 302, both the truncated linker 206 and scar 208 may be double-stranded oligonucleotides. These double-stranded oligonucleotides may be denatured and doing so will release single-strand fragments of the linker complement strand 304 leaving the truncated linker 206 and the scar 208 as both single-stranded oligonucleotides.

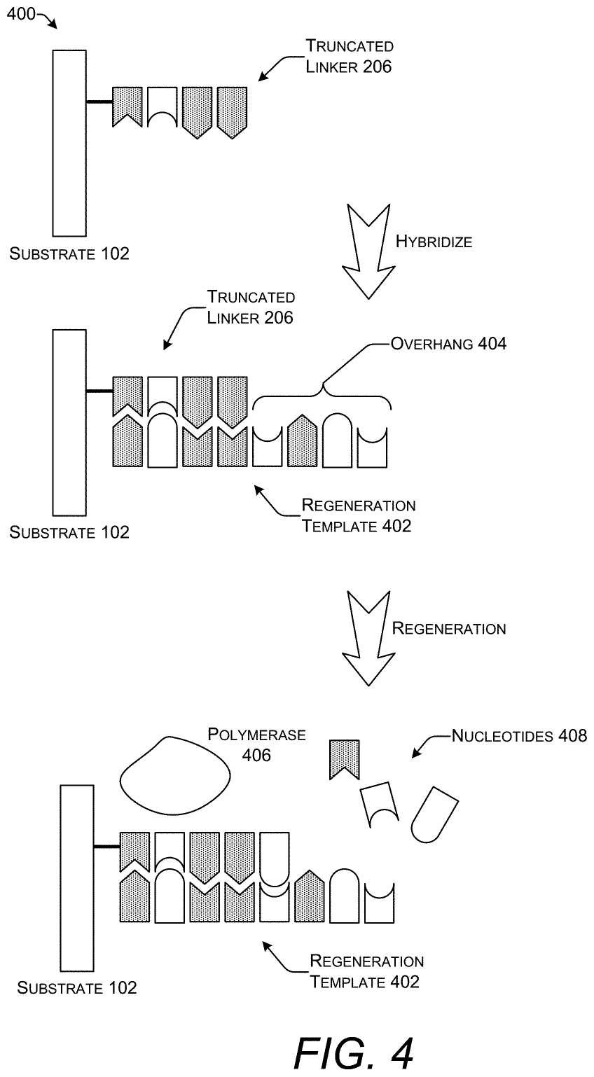

FIG. 4 shows an example time series 400 of regenerating a truncated linker 206 using polymerase 406 and nucleotides 408. A regeneration template 402 hybridizes to the truncated linker 206. The regeneration template 402 is a single-stranded oligonucleotide that is complementary to the full length of the single-stranded oligonucleotide linker 110. By hybridizing to the truncated linker 206, the regeneration template 402 creates an overhang 404 that serves as a template for regeneration of the full length of the single-stranded oligonucleotide linker 110. The regeneration template 402 may be the same as the linker complement strand 304 introduced in FIG. 3. However, in some implementations, the linker complement strand 304 may be shorter than the full length of the single-stranded oligonucleotide linker 110.

The truncated linker 206 and the regeneration template 402 are combined with polymerase 406 and nucleotides 408 under conditions that allow the polymerase 406 to add nucleotides 408 complementary to the overhang 404. The portion of the single-stranded oligonucleotide linker 110 that was cleaved off by the linker cleavage agent 204 is regenerated by the action of polymerase 406 adding the nucleotides 408 to the 3′-end of the truncated linker 206. However, in some implementations, the overhang 404 of the regeneration template 402 may have a sequence that is not complementary to the original single-stranded oligonucleotide linker 110. Thus, nucleotides 408 which are incorporated will be complementary to the overhang 404 and not reproduce the sequence of the original single-stranded oligonucleotide linker 110. This provides the ability to at least partially change the recognition site on a linker in situ. The type of polymerase 406 and nucleotides 408 may be selected based on the type of oligonucleotide used for the linker 110. For DNA, the polymerase 406 may be a DNA polymerase and nucleotides 408 may be deoxyribose nucleotide triphosphates (dNTPs). For RNA, the polymerase 406 may be an RNA polymerase and the nucleotides 408 may be nucleotide triphosphates (NTPs). Techniques for synthesizing a complementary strand of an oligonucleotide using polymerase 406 and nucleotides 408 are well-known to those of ordinary skill in the art.

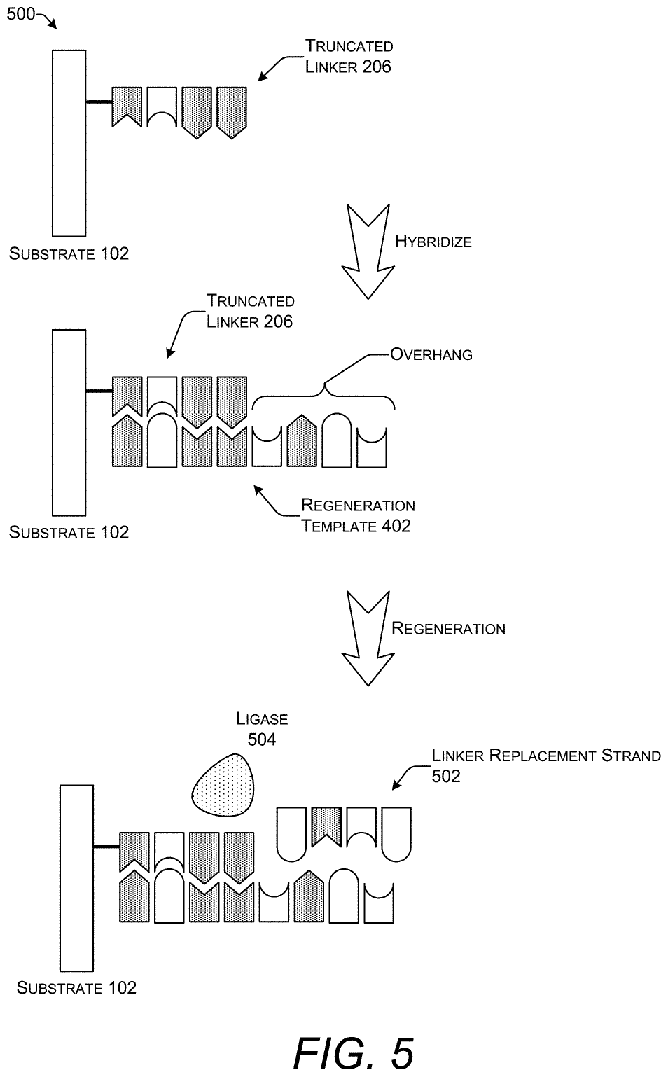

FIG. 5 shows an example time series 500 of regenerating a truncated linker 206 using a linker replacement strand 502 and ligase 504. This is an alternative to the technique shown in FIG. 4. As shown in FIG. 4, the regeneration template 402 hybridizes to the truncated linker 206 creating an overhang 404. However, rather than providing individual nucleotides 408, a linker replacement strand 502 is used to replace the nucleotides cleaved off by the linker cleavage agent 204.

The linker replacement strand 502 is a single-stranded oligonucleotide that may have the same sequence as the portion of the single-stranded oligonucleotide 110 that was cleaved off. Thus, the linker replacement strand 502 hybridizes to the overhang 404 of the regeneration template 402. In some implementations, the linker replacement strand 502 may have a different sequence than the portion of the single-stranded oligonucleotide linker 110 that was cleaved off. In such implementations, the overhang region of the regeneration template 402 would then be complementary to this different sequence rather than to the original single-stranded oligonucleotide linker 110. This provides the ability to at least partially change the recognition site on a linker in situ. The linker replacement strand 502 may be pre-synthesized using any conventional technique for generating short oligonucleotides. Ligase 504 is used to create a phosphodiester bond and seal the nick between the linker replacement strand 502 and the free end of the truncated linker 206. One example of a suitable ligase 504 is T4 DNA Ligase. The truncated linker 206 may be oriented so that its free end is either the 5′-end or the 3′-end. The double-stranded oligonucleotide may be denatured separating the regeneration template 402 which can then be washed away. Doing so regenerates a single-stranded oligonucleotide with the same sequence as the original single-stranded oligonucleotide linker 110.

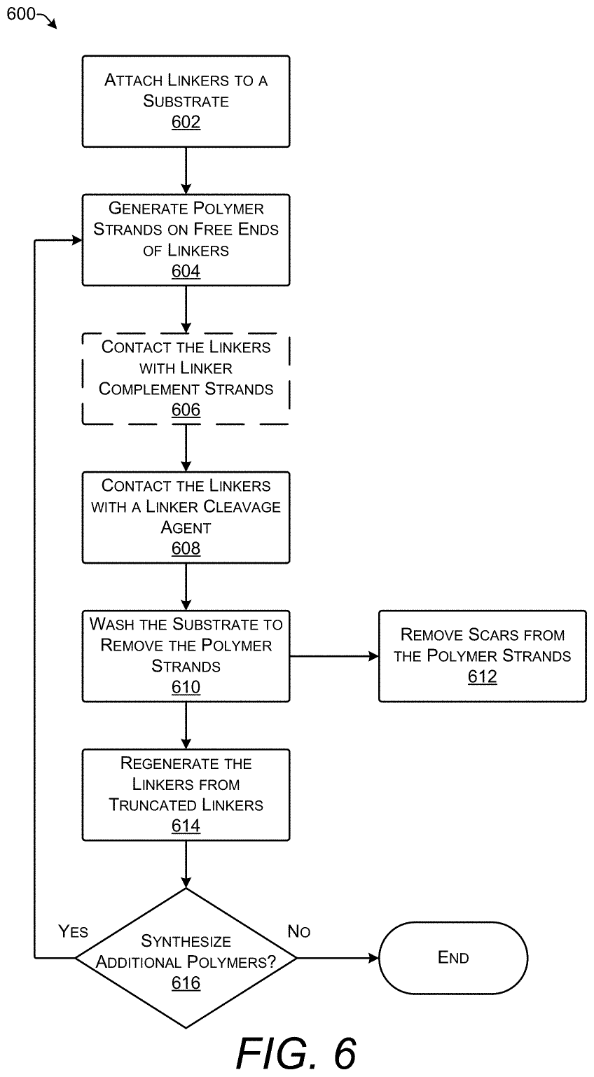

FIG. 6 shows an example process 600 for solid-phase polymer synthesis that releases polymer strands from a substrate by cleaving linkers and then regenerating the linker so that the substrate can be used again. Examples of portions of process 600 are also illustrated by the time series shown in FIGS. 2-5. Detail of procedures and techniques not explicitly described in this or other processes disclosed of this application are understood to be performed using conventional molecular biology techniques and knowledge readily available to one of ordinary skill in the art. Specific procedures and techniques may be found in reference manuals such as, for example, Michael R. Green & Joseph Sambrook, Molecular Cloning: A Laboratory Manual, Cold Spring Harbor Laboratory Press, 4^{th} ed. (2012).

At operation 602, linkers are attached to a substrate. The linkers may be attached to the substrate using any conventional technique for covalently or otherwise anchoring linker molecules to a solid substrate. The substrate, as prepared with the attached linkers, may be reused during multiple rounds of solid-phase polymer synthesis according to the techniques of this disclosure.

In one implementation, the linkers are polypeptide linkers. Polypeptide linkers are peptide chains of any length. In some implementations, each polypeptide linker includes about 5-10 amino acids which may have specific, pre-determined peptide sequences. The polypeptide linkers may be created from amino acids with non-reactive side groups to minimize the formation of branched structures. For example, amino acids used in the peptide linkers may be selected from the group of nonpolar, aliphatic amino acids that includes lysine, alanine, valine, lencine, methionine, and isoleucine. More specifically, the amino acids used in the peptide linkers may be one of glycine, alanine, or valine. For example, the polypeptide linkers may be glycine chains that include 5-8 amino acids.

In one implementation, the linkers are single-stranded oligonucleotide linkers. The single-stranded oligonucleotide linkers are single-stranded DNA, RNA, or hybrid DNA-RNA molecules. In some implementations, the oligonucleotide linkers are about 10-20 base pairs long. The oligonucleotide linkers may be created with specific, pre-determined sequences of nucleotides. The single-stranded oligonucleotide linkers may be oriented with respect to the substrate so that either the 3′-end or the 5′-end is attached to the substrate.

The substrate is a solid object having one or more rigid surfaces. The substrate can have any size and shape such as generally spherical beads or a generally flat “chip.” The substrate may be part of a larger object formed from the same or different material. The solid substrate can be comprised of any of glass, a silicon material, a metal material, plastic, or a combination thereof.

At operation 604, polymer strands are generated by adding monomers to the free ends of the linkers attached to the substrate. The polymer strands may be generated by any solid-phase synthesis technique suitable for generating the desired type of polymer. Those techniques may include phosphoramidite DNA synthesis, enzymatic DNA synthesis, and SPPS. The polymer strand may, in some implementations, be created by oligonucleotide assembly techniques that hybridize multiple pre-synthesized oligonucleotide units together to create an oligonucleotide chain with a specific sequence.

At operation 606, the linkers may be optionally contacted with linker complement strands under conditions that cause the linker complement strands to hybridize with the linkers. It is to be understood that the linker complement strands need not be 100% complementary to the linkers in order to hybridize. The linker complement strands are single-stranded oligonucleotides that are complementary to all or a portion of a single-stranded oligonucleotide linker. The linker complement strand may, at a minimum, be complementary to a recognition site in the single-stranded oligonucleotide linker that is cleaved by a linker cleavage agent. Linker complement strands may be used if the linker is a single-stranded oligonucleotide and the linker cleavage agent is an enzyme that recognizes double-stranded oligonucleotide sequences. If the linker is a polypeptide or the linker cleavage agent cleaves single-stranded oligonucleotides, addition of linker complement strands may be omitted.

At operation 608, the linkers are contacted with a linker cleavage agent that cleaves the linkers at a recognition site. Cleavage releases the polymer strands from the substrate and leaves truncated linkers in place of the original full-length linkers. The linkers may be contacted with the linker cleavage agent for a sufficient duration of time and under sufficient conditions (e.g., temperature) for the linker cleavage agent to cleave all or almost all of the linkers attached to the substrate. Specificity of the linker cleavage agent for a recognition site may limit which linkers are cleaved if a substrate is coated with multiple different types of linkers.

The linker cleavage agent may be a protease that cleaves polypeptides, a restriction endonuclease that cleaves oligonucleotides, or a non-enzymatic chemical cleavage agent. A protease may recognize an amino acid sequence in a polypeptide linker and break a peptide bond at a specific location. A chemical cleavage agent can also recognize a specific amino acid sequences and break a peptide bond at a specific location. A restriction endonuclease may recognize a nucleic acid sequence in an oligonucleotide linker and break a phosphodiester bond at a specific location.

At operation 610, the substrate is washed to remove the polymer strands. The specific technique used for washing the substrate depends on the structure of the substrate. A substrate structured as a generally flat chip or array may be washed by flowing a wash buffer across its surface. A substrate that is structured as small beads may be washed by eluting a wash buffer through a column containing the beads. Once the polymer strands are freed from the substrate they may be collected and purified, sequenced, analyzed, or otherwise used.

The wash buffer, elution buffer, or other fluid used to wash the polymer strands from the surface of the substrate may be aqueous or an organic solution depending on the protocols used for other steps in process 600. In some implementations, the wash buffer may simply be water. If the polymer strands are oligonucleotides any wash buffer suitable for washing or manipulating oligonucleotides such as TE, TAE, and TBE may be used. The washing stage may also include heating (e.g. to about 95° C.), or the wash buffer may be supplied heated, to promote disassociation of double-stranded oligonucleotides. The wash buffer may be an aqueous buffer solution or mixed aqueous/organic solvent. Examples of organic solvents that may be added to a wash buffer include polar, miscible organic cosolvents (e.g., DMSO, acetonitrile, etc.) which may be helpful in removing metal ions, organic residues, and denatured protein.

At operation 612, scars may be removed from the polymer strands. The scars are the remnants of the linkers that remain attached to the polymer strands after cleavage of the linkers at operation 608. If the linkers are made from different polymers than the polymer strands that were generated, selective digestion techniques may be used to remove the scars. For example, if the linkers are polypeptides and the polymer strands are oligonucleotides, proteases that digest polypeptides without affecting oligonucleotides may be used to remove the scar.

If the polymer strands are oligonucleotides, PCR may be used to amplify a desired portion of the polymer strand (e.g., a portion that does not include the scar). This will create many copies of the polymer strands that do not include the scar. Thus, the scar will be effectively removed from the polymer strands as a group because polymer strands that still have the scar attached will become greatly diluted by a large number of polymer strands that do not include the scar.

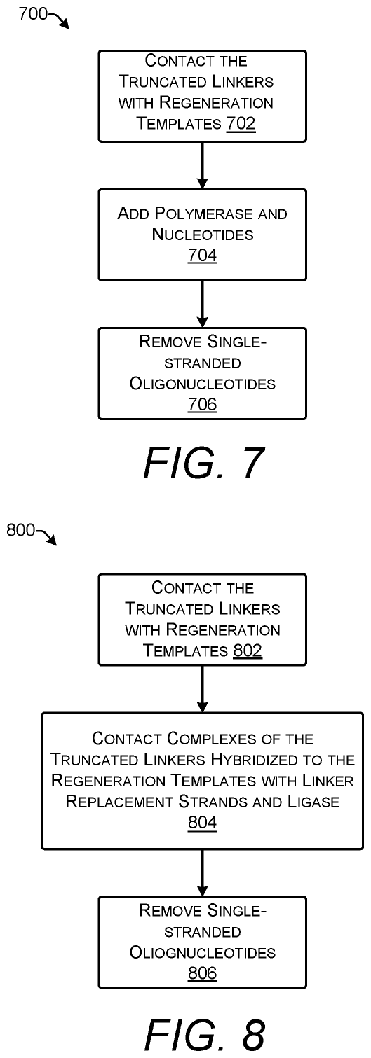

At operation 614, the linkers are regenerated from the truncated linkers created at operation 608. The specific technique used to regenerate a truncated linker depends in part on the type of linker. For polypeptide linkers, SPPS may be used to extend the truncated linkers by adding back the same sequence of peptides that was cleaved off. For single-stranded oligonucleotide linkers, some example regeneration techniques are discussed below and shown in FIG. 7 and FIG. 8. Once the linkers have been regenerated, the substrate is ready to be used again for synthesizing a different batch of polymer strands. Each batch of polymer strands synthesized on a substrate may be the same or different as the polymer strands synthesized in any other batch.

At operation 616, if the substrate is used to synthesize additional polymers then process 600 proceeds along the “yes” path and returns to operation 604 where new polymer strands are generated on the regenerated linkers. The regenerated linkers may be chemically and structurally identical to the original linkers so process 600 can continue through multiple iterations. If no more polymers are to be synthesized, then process 600 proceeds along the “no” path and ends. The substrate with the regenerated linkers may be stored and used again at a later time.

FIG. 7 shows an example process 700 for regenerating single-stranded oligonucleotide linkers using polymerase and nucleotides. Process 700 may be implemented in part using the time series shown in FIG. 4.

At operation 702, the truncated linkers are contacted with regeneration templates under conditions that cause the regeneration templates to hybridize with the truncated linkers. The regeneration template is a single-stranded oligonucleotide that is complementary to the full length of the single-stranded oligonucleotide linker. By hybridizing to the truncated linker, the regeneration template creates an overhang that serves as a template for regeneration of the full length of the single-stranded oligonucleotide linker.

At operation 704, the truncated linkers are extended by addition of polymerase and nucleotides. The polymerase adds the nucleotides onto the free end of the truncated linker using the regeneration template as a template to direct which nucleotides are added. This re-creates the same sequence of nucleotides that was cleaved off from the end of the linker. This operation regenerates a single-stranded oligonucleotide with the same sequence as the original single-stranded oligonucleotide linker.

At operation 706, a clean-up step may be performed in which single-stranded oligonucleotides are removed. Once the polymerase is finished adding nucleotides to the end of the truncated linker, there should be a double-stranded oligonucleotide structure attached to the surface of the substrate. One of the strands is the single-stranded oligonucleotide linker that was regenerated and the other strand is the regeneration template. However, there may be some locations at which the regeneration template does not anneal, polymerase does not add nucleotides, or something else prevents correct regeneration of the linker.

For these are other reasons there may be single-stranded oligonucleotides present on the surface of the substrate. To prevent the single-stranded oligonucleotides from interfering with subsequent rounds of solid-phase polymer synthesis, single-stranded oligonucleotides may be removed. Selective digestion of single-stranded oligonucleotides may be accomplished by addition of an exonuclease that is specific to single-stranded oligonucleotides such as Mung Bean nuclease. The single-stranded oligonucleotide linker is protected at this stage because it is still annealed to the regeneration template.

FIG. 8 shows an example process 800 for regenerating single-stranded oligonucleotide linkers using linker replacement strands and ligase. Process 800 may be implemented in part in the time series shown in FIG. 5.

At operation 802, the truncated linkers are contacted with regeneration templates under conditions that cause the regeneration templates to hybridize with the truncated linkers. The regeneration template is a single-stranded oligonucleotide that is complementary to the full length of the single-stranded oligonucleotide linker. By hybridizing to the truncated linker, the regeneration template creates an overhang that serves as a template for regeneration of the full length of the single-stranded oligonucleotide linker.

At operation 804, complexes of the truncated linkers hybridized to regeneration templates are contacted with linker replacement strands and ligase. The linker replacement strands are single-stranded oligonucleotides that can have the same sequence as the portion of the single-stranded oligonucleotide that was cleaved off. Thus, the linker replacement strands hybridize to the overhangs of the regeneration templates. The ligase creates a phosphodiester bond between the linker replacement strands and the truncated linkers. This creates an oligonucleotide strand with the same sequence as the original single-stranded oligonucleotide linker.

At operation 806, a clean-up step may be performed in which single-stranded oligonucleotides are removed. Once the polymerase is finished adding nucleotides to the end of the truncated linker, there should be a double-stranded oligonucleotide structure attached to the surface of the substrate. One of the strands is the single-stranded oligonucleotide linker that was regenerated and the other strand is the regeneration template. However, there may be some locations at which the regeneration template does not anneal, polymerase does not add nucleotides, or something else prevents correct regeneration of the linker.

For these are other reasons there may be single-stranded oligonucleotides present on the surface of the substrate. To prevent the single-stranded oligonucleotides from interfering with subsequent rounds of solid-phase polymer synthesis, single-stranded oligonucleotides may be removed. Selective digestion of single-stranded oligonucleotides may be accomplished by addition of an exonuclease that is specific to single-stranded oligonucleotides such as Mung Bean nuclease. The single-stranded oligonucleotide linker is protected at this stage because it is still annealed to the regeneration template.

### Cleavage at the Point of Attachment Between Linker and Polymer Strand

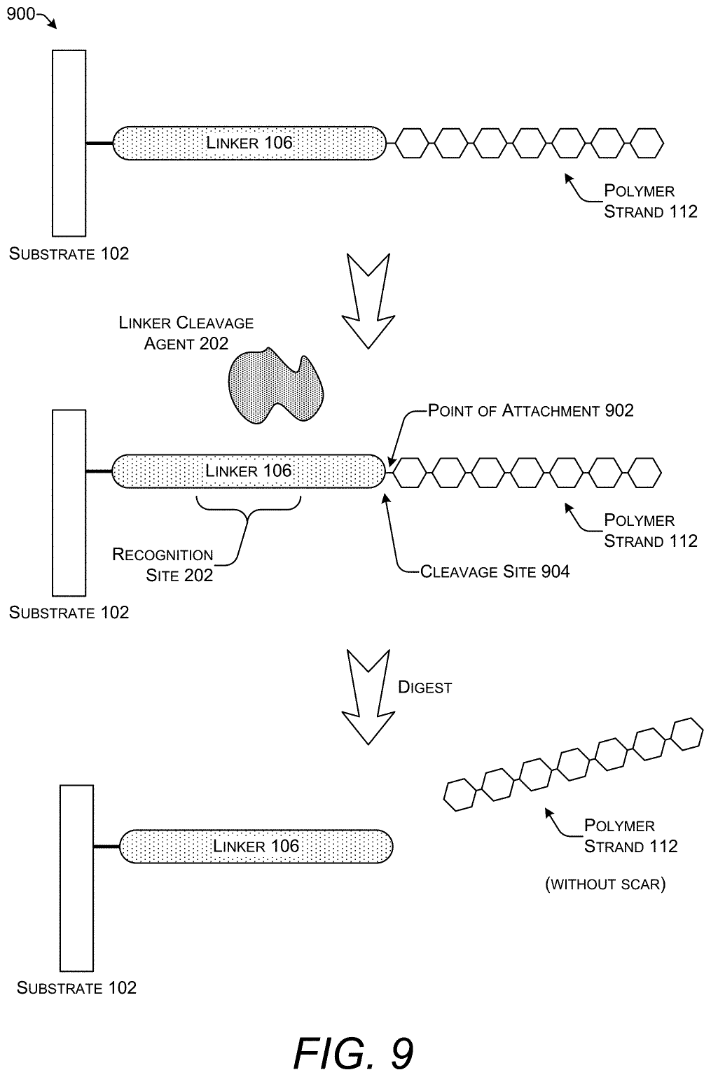

FIG. 9 shows an example time series 900 of cleaving a linker 106 at its point of attachment 902 to a polymer strand 112. In various implementations, the linker 106 may be either a polypeptide linker 108 or a single-stranded oligonucleotide linker 110. Cleaving the linker 106 precisely at the point of attachment 902 to the polymer strand 112 can avoid the need to regenerate a truncated linker 206 and releases the polymer strand 112 without scar 208. Selection of the linker cleavage agent 204 and the design of the linker 106 may both be modified to achieve cleavage at the point of attachment 902 rather than within the linker 106.

In one implementation, cleavage at the point of attachment 902 is achieved by using a linker cleavage agent 204 that is a restriction endonuclease with a cleavage site 904 outside of its recognition site 204. The restriction endonuclease cleaves the phosphodiester bond between the end of a single-stranded oligonucleotide linker 110 and the attached polymer strand 112.

Restriction endonucleases with these characteristics are referred to as type IIS restriction endonucleases. Many different type IIS restriction endonucleases are available from commercial sources. One example is BseGI (BtsCI) for which the recognition site 202 is GGATG and the cleavage site 904 site is two nucleotides to the 3′ side the final G (i.e., GGATGNN↓). All nucleotide sequences included in this disclosure are written in the 5′ to 3′ direction unless indicated otherwise. Thus, for example, a single-stranded oligonucleotide linker 110 with the structure ACGGATGCC-polymer would be cleaved by BseGI after the final C at the point of attachment 902 to the polymer strand 112. Another example is BsgI which has a recognition site 202 of GTAGCAG and a cleavage site 904 sixteen nucleotides to the 3′ end of the final G (i.e., GTAGCAG(N)₁₆↓).

In one implementation, single-stranded oligonucleotide linkers 110 that include both DNA and RNA use the ability of enzymes to recognize the difference in order to locate the cleavage site 904 at the very end of the linkers 110. This may be accomplished by using a uracil cleavage system in which the sequential addition of Uracil DNA Glycosylase (UDG) and endonuclease VIII generates a single nucleotide gap at the location of uracil bases in an oligonucleotide containing a deoxy-uracil. UDG catalyzes the excision of the uracil base, creating an abasic site with an intact phosphodiester backbone. The lyase activity of Endonuclease VII breaks the phosphodiester backbone both 3′ and 5′ to the abasic site, liberating the deoxyribose sugar.

For example, a single-stranded oligonucleotide linker 110 with the sequence AGCTAGCTU after digestion with UDH and endonuclease VIII is reduced to AGCTAGCT and the polymer strand 112 attached to the uracil is released without a scar. Single-stranded oligonucleotide linkers 110 designed to work with the system may include only a single uracil at the free end in order to prevent cleavage within the middle of the linker 110. Because the uracil is released by cleavage of the phosphodiester backbone on both the 3′ and 5′ ends, the single-stranded oligonucleotide linker 110 may be regenerated by addition of a single uracil. Thus, this technique cleaves at the point of attachment 902 but also regenerates the linker 110 after each round of solid-phase polymer synthesis.

Polypeptide linkers 108 may be cleaved at the point of attachment 902 by design of a peptide sequence and selection of a protease such that the location where the protease cleaves the peptide bonds at the point of attachment 902. For example, a polypeptide linker 108 may be designed so that the peptide at its free end 116 is Asp, the free end 116 is the N-terminal, and there are no other aspartic acid residues in the polypeptide linker 108. Use of AspN as the linker cleavage agent 202 would then cleave at the end of the polypeptide linker 108 which is the N-terminal of Asp.

Peptide-nucleotide bonds may be cleaved by enzymes that recognize the peptide-nucleotide bond such as the 23S ribosomal RNA from Thermos aquaticus. See Xiaochang Dai et al., Cleavage of an Amide Bond by a Ribozyme, 267 Science 237 (1995) for a discussion of cleavage of amide linkages between DNA and amino acids or short peptides. Thus, polypeptide linker 108 used for synthesis of oligonucleotides may be cleaved that the point of attachment 902 by a ribozyme such as 23S. Similarly, single-stranded oligonucleotide linkers 110 used synthesize polypeptides may also be cleaved at the point of attachment 902 by cleaving the peptide-nucleotide bond.

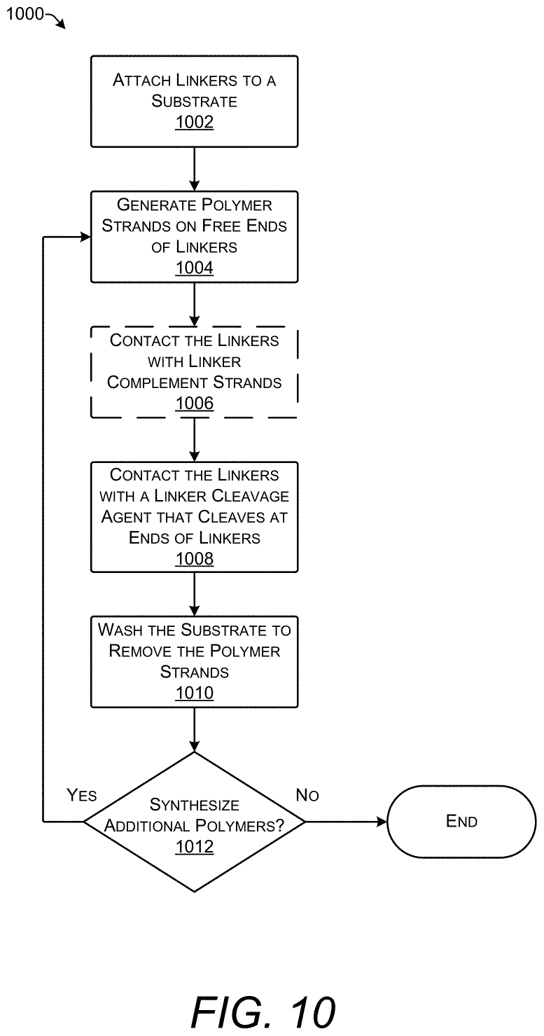

FIG. 10 shows an example process 1000 for solid-phase polymer synthesis that releases polymer strands from a substrate by cleaving at the point of connection between linkers and the polymer strands. Examples of portions of process 1000 are also illustrated by the time series shown in FIG. 9.

At operation 1002, linkers are attached to a substrate. The linkers may be attached to the substrate using any conventional technique for covalently or otherwise anchoring linker molecules to a solid substrate. The substrate, as prepared with the attached linkers, may be reused during multiple rounds of solid-phase polymer synthesis according to the techniques of this disclosure.

In one implementation, the linkers are polypeptide linkers. Polypeptide linkers are peptide chains of any length. In some implementations, each polypeptide linker includes about 5-10 amino acids which may have specific, pre-determined peptide sequences. The polypeptide linkers may be created from amino acids with non-reactive side groups to minimize the formation of branched structures. For example, amino acids used in the peptide linkers may be selected from the group of nonpolar, aliphatic amino acids that includes alanine, valine, leucine, methionine, proline, and isoleucine. More specifically, the amino acids used in the peptide linkers may be one of glycine, alanine, or valine. For example, the polypeptide linkers may be glycine chains that include 5-8 amino acids.

However, the polypeptide linkers may include amino acids with reactive side groups that are recognized by proteases which cleave the polypeptide linkers. To minimize the possibility of off-target cleavage, amino acids in a polypeptide linker other than at an enzyme recognition site may be selected from the group of nonpolar, aliphatic amino acids. For example, amino acids positioned between the substrate and a cleavage site may be selected from the group of nonpolar, aliphatic amino acids and amino acids in the cleavage site may include reactive side groups.

In one implementation, the linkers are single-stranded oligonucleotide linkers. The single-stranded oligonucleotide linkers are single-stranded DNA, RNA, or hybrid DNA-RNA molecules. In some implementations, the oligonucleotide linkers are about 10-20 base pairs long. The oligonucleotide linkers may be created with specific, pre-determined sequences of nucleotides. The single-stranded oligonucleotide linkers may be oriented with respect to the substrate so that either the 3′-end or the 5′-end is attached to the substrate.

The substrate is a solid object having one or more rigid surfaces. The substrate can have any size and shape such as generally spherical beads or a generally flat “chip.” The substrate may be part of a larger object formed from the same or different material. The solid substrate can be comprised of any of glass, a silicon material, a metal material, plastic, or a combination thereof.

At operation 1004, polymer strands are generated by adding monomers to free ends of linkers attached to the substrate. The polymer strands may be generated by any solid-phase synthesis technique suitable for generating the desired type of polymer. Those techniques may include phosphoramidite DNA synthesis, enzymatic DNA synthesis, and SPPS. The polymer strand may, in some implementations, be created by oligonucleotide assembly techniques that hybridize multiple pre-synthesized oligonucleotide units together to create an oligonucleotide chain with a specific sequence.

At operation 1006, the linkers may be optionally contacted with linker complement strands under conditions that cause the linker complement strands to hybridize with the linkers. It is to be understood that linker complement strands need not be 100% complementary to the linkers in order to hybridize. The linker complement strands are single-stranded oligonucleotides that are complementary to all or a portion of a single-stranded oligonucleotide linker. The linker complement strand may, at a minimum, be complementary to a recognition site in the single-stranded oligonucleotide linker that is cleaved by a linker cleavage agent. Linker complement strands may be used if the linker is a single-stranded oligonucleotide and the linker cleavage agent is an enzyme that recognizes double-stranded oligonucleotide sequences. If the linker is a polypeptide or the linker cleavage agent cleaves single-stranded oligonucleotides, addition of linker complement strands may be omitted.

At operation 1008, linkers are contacted with a linker cleavage agent that cleaves the linkers at the points of attachment between the linkers and the polymer strands. Cleaving exactly at the point of attachment frees the polymer strands from the linkers without leaving a scar attached to the polymer strands. Also, because the linkers are not cleaved, the full-length linkers remain and it is not necessary to regenerate portions of the linkers.

At operation 1010, the substrate is washed to remove the polymer strands without removing the linkers from the substrate. The specific technique used for washing the substrate depends on the structure of the substrate. A substrate structured as a generally flat chip or array may be washed by flowing a wash buffer across its surface. A substrate that is structured as small beads may be washed by eluting a wash buffer through a column containing the beads. Once the polymer strands are freed from the substrate they may be collected and purified, sequenced, analyzed, or otherwise used.

At operation 1012, if the substrate is used to synthesize additional polymers then process 1000 proceeds along the “yes” path and returns to operation 1004 where new polymer strands are generated on the linkers. The free ends of the linkers may be chemically modified so that the linkers are again able to bond to monomers of the polymer strands. For example, if the linkers are cleaved using UDG, the linkers may be prepared for reuse by adding uracil nucleotides to their free ends. If no more polymers are to be synthesized, then process 1000 proceeds along the “no” path and ends. The substrate with the linkers may be stored and used again at a later time.

### Denaturation of Linkers

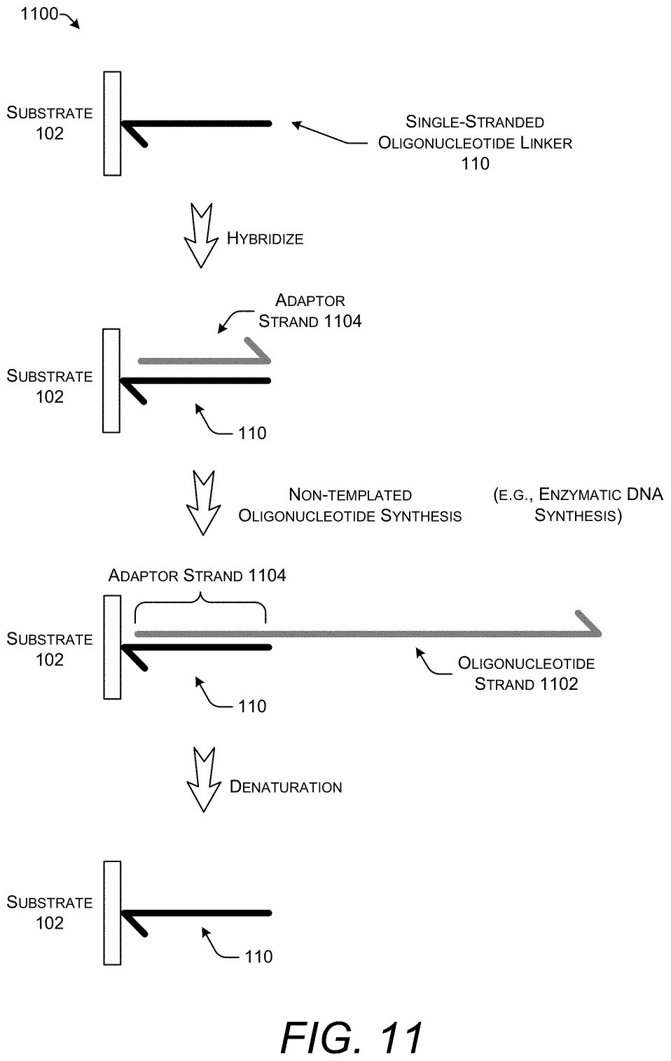

FIG. 11 shows an example time series 1100 of using a single-stranded oligonucleotide linker 110 with its 3′-end attached to the substrate 102 as the linker for synthesis of an oligonucleotide strand 1102. An adapter strand 1104 is hybridized to the single-stranded oligonucleotide linker 110. The adapter strand 1104 is also a single-stranded oligonucleotide. The adapter strand 1104 may be the same length as the single-stranded oligonucleotide linker 110 or it may be slightly shorter such that it does not extend all the way to the base of the single-stranded oligonucleotide linker 110. Thus, some nucleotides at the 3′-end of the single-stranded oligonucleotide linker 110 are not hybridized to the adapter strand 1104. Hybridization of the adapter strand 1104 to the single-stranded oligonucleotide linker 110 creates a “blunt-ended” double-stranded oligonucleotide structure attached to the surface of the substrate 102.

The adapter strand 1104 is extended from its 3′-end to create the oligonucleotide strand 1102. Because there is no template from which to build the oligonucleotide strand 1102, a non-templated oligonucleotide synthesis technique is used. One example of a non-templated oligonucleotide synthesis technique is enzymatic synthesis with TdT. TdT adds nucleotides in a 3′ to 5′ direction and is thus able to begin synthesis from the 3′-end of the adapter strand 1104.

Extension of the adapter strand 1104 creates the oligonucleotide strand 1102. The oligonucleotide strand 1102 includes nucleotides of the adapter strand 1104 and those nucleotides subsequently added through non-templated oligonucleotide synthesis.

The oligonucleotide strand 1102 is separated from the single-stranded oligonucleotide linker 110 simply by denaturation. Denaturation breaks the hydrogen bonds holding the adapter strand 1104 to the single-stranded oligonucleotide linker 110 and releases the oligonucleotide strand 1102. The denaturation may be initiated by any suitable technique including, but not limited to, heating above the Tₘ of the assembled double-stranded oligonucleotide, adding sodium hydroxide, and/or decreasing the salt concentration. These and other techniques for denaturing double-stranded oligonucleotides are well-known to those of ordinary skill in the art.

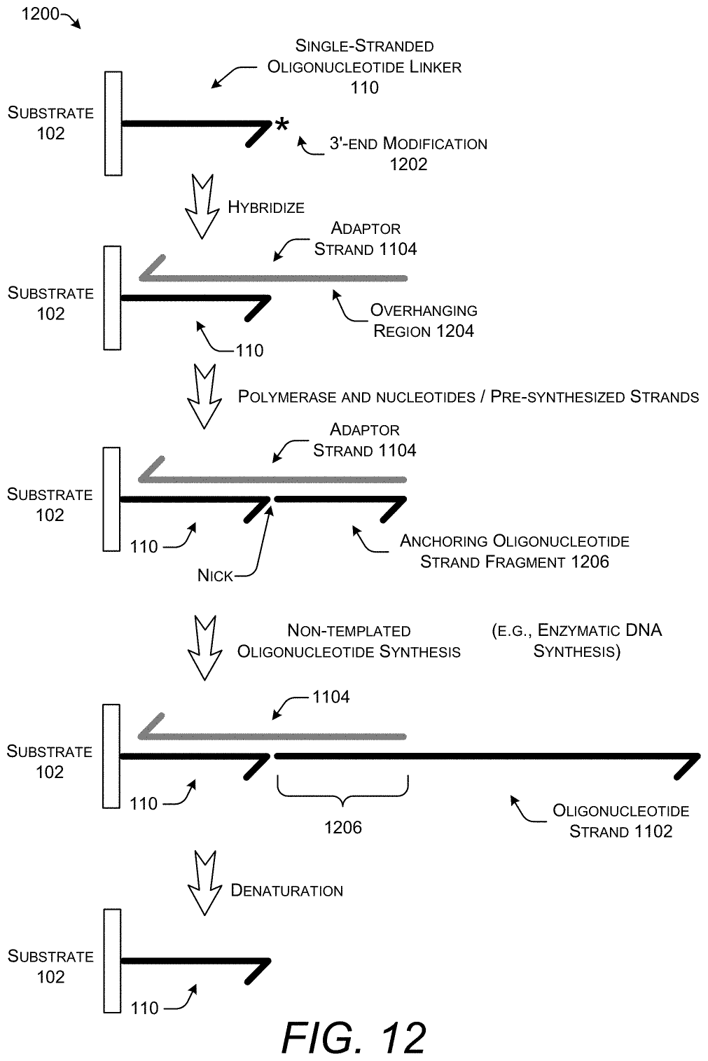

FIG. 12 shows an example time series 1200 of generating an oligonucleotide strand 1102 using a single-stranded oligonucleotide linker 110 with its 5′-end attached to the substrate 102. The single-stranded oligonucleotide linker 110 may have a 3′-end modification 1202. The 3′-end modification 1202 prevents extension of the single-stranded oligonucleotide linker 110. In one implementation, the 3′-end modification 1202 is incorporation of nucleoside analog as the final nucleotide at the 3′-end of the single-stranded oligonucleotide linker 110. One type of nucleoside analog that prevents chain extension is des-hydroxy dNTP (or NTP for RNA) which does not have an available 3′ OH group.

An adapter strand 1104 is hybridized to the single-stranded oligonucleotide linker 110. The adapter strand 1104, in this implementation, is a single-stranded oligonucleotide that is longer than the single-stranded oligonucleotides linker 110 and forms an overhanging region 1204. The length of the overhanging region 1204 may be varied considerably without affecting the results. In some implementations, the length of the overhanging region 1204 may be the same as or similar to the length of the single-stranded oligonucleotide linker 110. For example, the length of the overhanging region 1204 may be about 5-15 base pairs.

An anchoring oligonucleotide strand fragments 1206 is hybridized to the overhanging region 1204 of the adapter strand 1104. However, the 3′-end modification 1202 prevents the anchoring oligonucleotide strand fragments 1206 from forming a covalent bond to the single-stranded oligonucleotide linker 110.

In one implementation, the anchoring oligonucleotide strand fragments 1206 are synthesized in situ by using polymerase to add nucleotides complementary to the overhanging region 1204 of the adapter strand 1104. In this implementation, the 3′-end modification 1202 may consist of omitting a final phosphate group from the nucleotide at the 3′-end of the single-stranded oligonucleotide linker 110. This prevents polymerase from forming a phosphodiester bond between the end of the single-stranded oligonucleotide linker 110 and the nucleotides added to create the anchoring oligonudeotide strand fragments 1206.

In one implementation, the anchoring oligonucleotide strand fragments 1206 may a pre-synthesized oligonucleotide strand that is complementary to the overhanging region 1204 of the adapter strand 1104. The anchoring oligonucleotide strand fragments 1206 may be synthesized by any conventional technique for generating short oligonucleotides.

After the anchoring oligonucleotide strand fragments 1206 has hybridized to the overhanging region 1204 of the adapter strand 1104, there is a “blunt-ended” double-stranded oligonucleotide structure attached to the surface of the substrate 102. The 3′-end of the anchoring oligonucleotide strand fragment 1206 is extended by a non-templated oligonucleotide synthesis technique to create the oligonucleotide strand 1102. One example of a non-templated oligonucleotide synthesis technique is enzymatic synthesis with TdT. TdT adds nucleotides in a 3′ to 5′ direction and is thus able to begin synthesis from the 3′-end of the anchoring oligonucleotide strand fragment 1206.

Extension of the anchoring oligonucleotide strand fragment 1206 creates the oligonucleotide strand 1102. The oligonucleotide 1002 includes nucleotides of the anchoring oligonucleotide strand fragment 1206 and those nucleotides subsequently added through non-templated oligonucleotide synthesis.

The oligonucleotide strand 1102 is separated from the single-stranded oligonucleotide linker 110 simply by denaturation. Denaturation breaks the hydrogen bonds holding the adapter strand 1104 to the single-stranded oligonucleotide linker 110 and to the oligonucleotide strand 1102. Because there is a nick in the oligonucleotide backbone between the single-stranded oligonucleotide linker 110 and the anchoring oligonucleotide strand fragment 1206, denaturation releases the oligonucleotide strand 1102 from the substrate 102. The denaturation may be initiated by any suitable technique including, but not limited to, heating above the Tₘ of the assembled double-stranded oligonucleotide, adding sodium hydroxide, and/or decreasing the salt concentration. These and other techniques for denaturing double-stranded oligonucleotides are well-known to those of ordinary skill in the art.

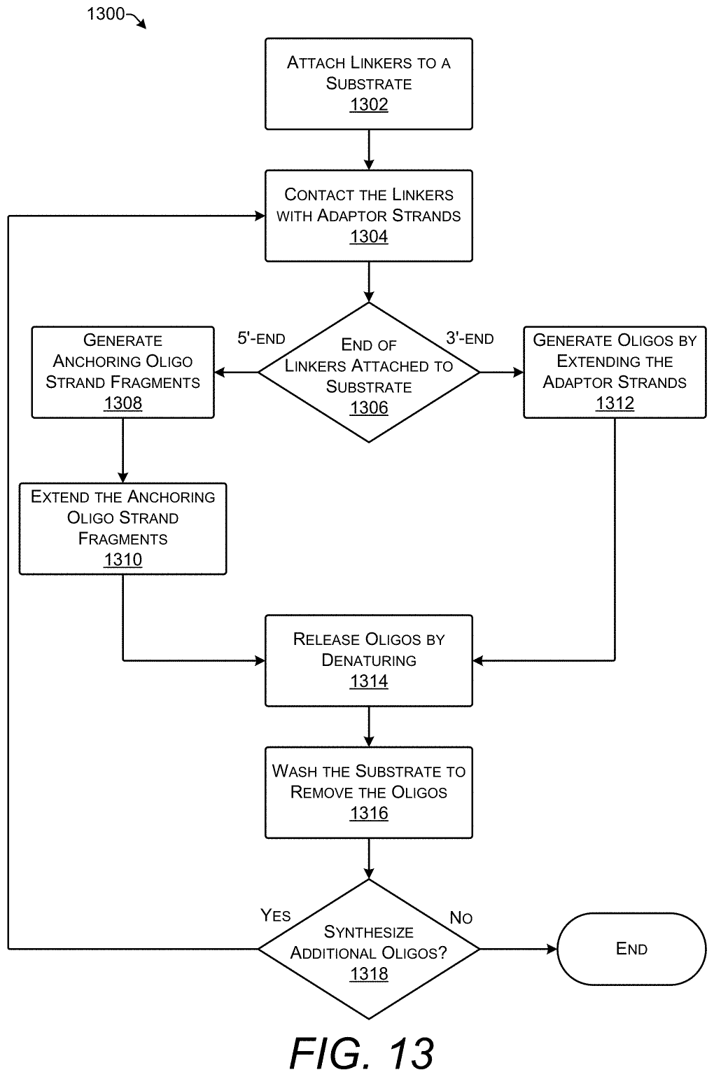

FIG. 13 shows an example process 1300 for solid-phase polymer synthesis that uses denaturation to release synthesized oligonucleotides from single-stranded oligonucleotide linkers. Examples of portions of process 1300 are also illustrated by the time series shown in FIGS. 11 and 12.

At operation 1302, single-stranded oligonucleotide linkers are attached to a substrate. The linkers may be attached to the substrate using any conventional technique for covalently or otherwise anchoring oligonucleotides to a solid substrate. The substrate, as prepared with the attached linkers, may be reused during multiple rounds of solid-phase polymer synthesis according to the techniques of this disclosure.

The single-stranded oligonucleotide linkers are single-stranded DNA, RNA, or hybrid DNA-RNA molecules. In some implementations, the oligonucleotide linkers are about 10-20 base pairs long. The oligonucleotide linkers may be created with specific, pre-determined sequences of nucleotides. The single-stranded oligonucleotide linkers may be oriented with respect to the substrate so that either the 3′-end or the 5′-end is attached to the substrate.

At operation 1304, the single-stranded oligonucleotide linkers are contacted with adapter strands under conditions that cause the adapter strands to hybridize with the oligonucleotide linkers. It is to be understood that adapter strands need not be 100% complementary to the oligonucleotide linkers in order to hybridize. The adapter strands are single-stranded oligonucleotides that form double-stranded structures with the linkers either with or without overhanging regions extending beyond the free end of the linkers.

At operation 1306, process 1300 diverges depending whether the end of the linkers attached to the substrate is the 5′-end or the 3′-end. If the 5′-ends of the linkers are attached to the substrate, and the 3′-end is the free end, process 1300 proceeds to operation 1308. If the 3′-end is attached to the substrate, and the 5′-end is the free end, process 1300 proceeds to operation 1312.

At operation 1308, anchoring oligonucleotide strand fragments are generated. The anchoring oligonucleotide strand fragments are single-stranded oligonucleotides complementary to the overhang region of the adapter strand. In one implementation, the anchoring oligonucleotide strand fragments are generated by contacting the adapter strands with pre-synthesized oligonucleotide strands under conditions that cause the pre-synthesized oligonucleotide strands to hybridize with the overhanging regions of the adapter strands.

In one implementation, the anchoring oligonucleotide strand fragments are generated by contacting the adapter strands with polymerase and nucleotides so that the adapter strands serve as templates for synthesis of the anchoring oligonucleotide strand fragments. The type of polymerase and nucleotides may be selected based on the type of oligonucleotide to be synthesized. For DNA, the polymerase may be a DNA polymerase and the nucleotides may be deoxyribose nucleotide triphosphates (dNTPs). For RNA, the polymerase may be an RNA polymerase and the nucleotides may be nucleotide triphosphates (NTPs). Techniques for synthesizing a complementary strand of an oligonucleotide using polymerase and nucleotides are well-known to those of ordinary skill in the art.

At operation 1310, the oligonucleotides are generated by extending the anchoring oligonucleotide strand fragments generated at operation 1308. The anchoring oligonucleotides fragments are extended from their 3′ ends by sequential addition of nucleotides using a non-templated oligonucleotide synthesis technique such as enzymatic synthesis with TdT. The anchoring oligonucleotide strand fragments once generated, are annealed to the overhang region of the adapter strand but not covalently connected to the single-stranded oligonucleotides linker. Thus, the oligonucleotide generated at operation 1310 is partially hybridized to the adapter strands.

Following the path of process 1300 in which the 3′ ends of the linkers are attached to the substrate, at operation 1312, the oligonucleotides are generated by extending the free 3′-ends of the adapter strands with a non-templated oligonucleotide synthesis technique. This creates oligonucleotides that include at the adapter strands at their 5′-ends. The adapter strands may later be removed from the oligonucleotides through enzymatic cleavage or PCR amplification.

At operation 1314, the oligonucleotides generated at operation 1310 or operation 1312 are released from the substrate by denaturing the single-stranded oligonucleotide linkers and the adapter strands. The double-stranded oligonucleotide structures of oligonucleotide linkers and adapter strands may be denatured by any suitable technique including, but not limited to, heating above the Tₘ of the assembled double-stranded oligonucleotide, adding sodium hydroxide, and/or decreasing the salt concentration. These and other techniques for denaturing double-stranded oligonucleotides are well-known to those of ordinary skill in the art.

Denaturing the double-stranded structure holding the synthesized oligonucleotides to the substrate releases the oligonucleotides without cleavage of any structure. Thus, the single-stranded oligonucleotides linkers remain attached to the substrate with the original configurations ready for use in a subsequent round of solid-phase polymer synthesis.

At operation 1316, the substrate is washed to remove the oligonucleotide strands without removing the single-stranded oligonucleotide linkers attached to the substrate. The specific technique used for washing the substrate depends on the structure of the substrate. A substrate structured as a generally flat chip or array may be washed by flowing a wash buffer across its surface. A substrate that is structured as small beads may be washed by eluting a wash buffer through a column containing the beads. Once the oligonucleotides are freed from the substrate they may be collected and purified, sequenced, analyzed, or otherwise used.

The wash buffer, elution buffer, or other fluid used to wash the oligonucleotides from the surface of the substrate may be aqueous or an organic solution depending on the protocols used for other steps in process 600. In some implementations, the wash buffer may simply be water. Any wash buffer suitable for washing or manipulating oligonucleotides such as TE, TAE, and TBE may be used. The wash buffer may be an aqueous buffer solution or mixed aqueous/organic solvent. Examples of organic solvents that may be added to a wash buffer include polar, miscible organic cosolvents (e.g., DMSO, acetonitrile, etc.) which may be helpful in removing metal ions, organic residues, and denatured protein.

At operation 1318, if the substrate is used to synthesize additional polymers then process 1300 proceeds along the “yes” path and returns to operation 1304 where the linkers are contacted with adapter strands. If no more polymers are to be synthesized, then process 1300 proceeds along the “no” path and ends. The substrate with the single-stranded oligonucleotide linkers may be stored and used again at a later time.

### Illustrative Patterned Substrate

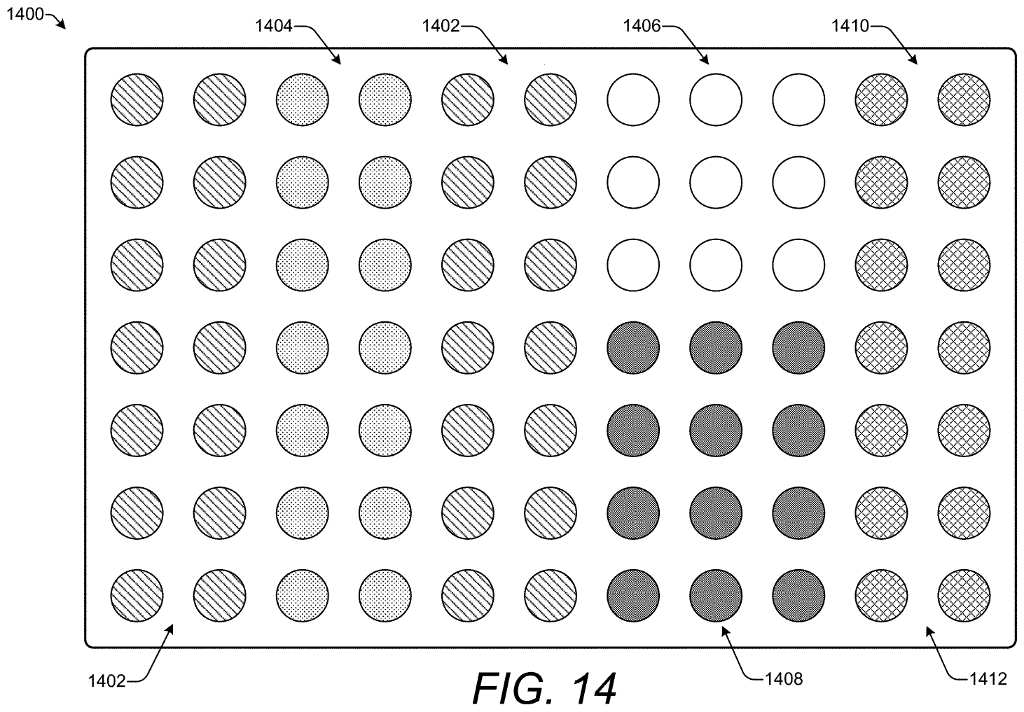

FIG. 14 shows one example of a patterned substrate 1400. The patterned substrate 1400 may be the same as any of the substrates 102 discussed in this disclosure. A substrate may be coated with many millions of individual linkers. All of the linkers attached to a substrate may be the same or a substrate may be patterned with different linkers placed at different locations. The linkers may be attached to the surface of the substrate 1400 using conventional surface functionalization chemistry techniques known to those of ordinary skill in the art.

There are many techniques suitable for creating groups of spatially-isolated linkers. Linkers could be positioned using site-selective chemistry such as by covering the surface of the substrate 1400 with protecting groups, removing the protecting groups at selected locations to expose a functional handle, and adding the linkers. Techniques for selective deblocking of protecting groups include use of photomasks and exposure to ultraviolet light, localized heating (e.g., using resistors embedded in the substrate 1400), and electrochemistry (e.g., using electrodes embedded in the substrate 1400 to control the location of chemical reactions). Linkers may also be synthesized directly at specific locations on the substrate 1400 using inkjet printing, phosphoramidite-based or enzymatic-based synthesis methods for oligonucleotides, or SPPS for polypeptides.

The illustrative patterned substrate 1400, includes five different groups of linkers 1402, 1404, 1406, 1408, 1410, and 1412. Each of the different groups of linkers 1402-1412 includes multiple clusters of linkers. Although a group may also include only a single cluster. Each of the different groups of linkers 1402-1412 represents a different type of linker providing for orthogonal cleavage and separation of polymer strands.

For example, linkers on the same substrate 1400 may be made from different types of molecules such as oligonucleotides and polypeptides. The linkers may also be made from the same type of molecule but differ in their sequences resulting in recognition by different enzymes (e.g., endonucleases or proteases). The linkers may be single-stranded oligonucleotide linkers that denature under different conditions (e.g., GC rich linkers denature at a higher temperature than AT-rich linkers) to create orthogonality based not on cleavage with an enzyme but on conditions for denaturation.

Patterning the surface of the substrate 1400 with groups of different types of linkers allows for random access to specific groups of synthesized polymer strands based on the differing cleavage modality of the linkers. One region, for example the group of linkers 1406, may be used for quality control. This group of linkers 1406 may be cleaved first and the polymer strands tested to determine if synthesis is complete and/or accurate.

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. A method for solid-phase synthesis of polymers, the method comprising: generating polymer strands (112) by adding monomers (114) to free ends of linkers (116) attached to a substrate (102); contacting the linkers (106) with a linker cleavage agent (204) that cleaves the linkers (106) at a recognition site (202) thereby releasing the polymer strands (112) from the substrate (102) and generating truncated linkers (206); and regenerating the linkers (106) from the truncated linkers (206).

Clause 2. The method of clause 1, wherein the linkers (106) comprise single-stranded oligonucleotide linkers (110) and the linker cleavage agent (204) comprises a restriction endonuclease that cleaves the linkers (110) within the recognition site (202).

Clause 3. The method of clause 2, further comprising, prior to contacting the linkers (110) with the linker cleavage agent (204), contacting the linkers (110) with linker complement strands (304) under conditions that cause the linker complement strands (304) to hybridize with the linkers (110), wherein the recognition site (202) is a double-stranded oligonucleotide sequence (302) formed from hybridization of the linkers (110) and the linker complement strands (304).

Clause 4. The method of any of clauses 1-3, wherein the linkers (106) comprise single-stranded oligonucleotide linkers (110) and regenerating the linkers (110) comprises: contacting the truncated linkers (206) with regeneration templates (402) under conditions that cause the regeneration templates (402) to hybridize with the truncated linkers (206); and (i) extending the truncated linkers (206) by addition of polymerase (406) and nucleotides (408); or (ii) contacting the regeneration templates (402) with linker replacement strands (502) and ligase (504).

Clause 5. The method of claim 1, wherein the linkers comprise polypeptide linkers (108) and the linker cleavage agent (202) comprises a protease that cleaves the linkers (108) at the recognition site (204).

Clause 6. The method of claim 1, wherein the linkers comprise polypeptide linkers (108) and the linker cleavage agent (202) comprises a chemical cleavage agent.

Clause 7. The method of any of clauses 1, 5, or 6, wherein the linkers comprise polypeptide linkers (108) and regenerating the linkers (108) comprises performing solid-phase synthesis of polypeptides to extend the truncated linkers (206).

Clause 8. The method of any of clauses 1-7, wherein the polymer strands (112) comprise oligonucleotides and the method further comprises removing scars (208) from the polymer strands (112) by amplification with polymerase chain reaction (PCR).

Clause 9. A method for solid-phase synthesis of polymers, the method comprising: a. generating polymer strands (112) by adding monomers (114) to free ends of linkers (106) attached to a substrate (102); b. contacting the linkers (106) with a linker cleavage agent (202) that cleaves at points of attachment (902) between the linkers (106) and the polymer strands (112); and c. washing the substrate (102) to remove the polymer strands (112) without removing the linkers (106) from the substrate (102).

Clause 10. The method of clause 9, further comprising repeating steps a-c at least once.

Clause 11. The method of any of clauses 9-10, wherein the linkers (106) comprise single-stranded oligonucleotide linkers (110) and the method further comprises, prior to contacting the linkers (106) with the linker cleavage agent (204), contacting the linkers (110) with linker complement strands (304) under conditions that cause the linker complement strands (302) to hybridize with the linkers (110).

Clause 12. The method of clause 11, wherein the linker cleavage agent (204) comprises a restriction endonuclease having a recognition site (202) within a double-stranded oligonucleotide sequence (302) formed by hybridization of the linkers (110) with the linker complement strands (304) and a cleavage site at the point of attachment (902) of the linkers (110) to the polymer strands (112).

Clause 13. The method of clause 11, wherein the linkers (106) comprise deoxyribonucleic acid (DNA) strands having uracil bases at the free ends (116) and the linker cleavage agent (202) comprises Uracil DNA Glycosylase (UDG).

Clause 14. The method of any of clauses 9-10, wherein the linkers comprise polypeptide linkers (108) and the linker cleavage agent (202) comprises a protease that cleaves at the points of attachment (902) between the polypeptide linkers (108) and the polymer strands (112).

Clause 15. A method of solid-phase synthesis of oligonucleotides, the method comprising: contacting single-stranded oligonucleotide linkers (110) attached to a substrate (102) with adapter strands (1004) under conditions that cause the adapter strands (1004) to hybridize with the single-stranded oligonucleotide linkers (110); generating oligonucleotide strands (1002) that are (i) covalently attached to the adapter strands (1004) or (ii) partially hybridized to the adapter strands (1004); and releasing the oligonucleotide strands (1002) from the substrate (102) by denaturing the single-stranded oligonucleotide linkers (110) and the adapter strands (1004).

Clause 16. The method of clause 15, wherein the single-stranded oligonucleotide linkers (110) are oriented with 3′-ends attached to the substrate (102) and free 5′-ends; and generating the oligonucleotide strands (1002) that are (i) covalently attached to the adapter strands (1004) comprises extending the adapter strands (1004) by non-templated oligonucleotide synthesis thereby creating the oligonucleotide strands (1002).

Clause 17. The method of clause 15, wherein the single-stranded oligonucleotide linkers (110) are oriented with 5′-ends attached to the substrate (102) and free 3′-ends, wherein the 3′-ends are modified (1102) to prevent extension, and generating the oligonucleotide strands (1002) (ii) partially hybridized to the adapter strands (1004) comprises: generating anchoring oligonucleotide strand fragments (1106) complementary to overhanging regions (1104) of the adapter strands (1004); and extending the anchoring oligonucleotide strand fragments (1106) by non-templated oligonucleotide synthesis thereby creating the oligonucleotide strands (1002).

Clause 18. The method of clause 17, wherein the generating the anchoring oligonucleotide stand fragments (1106) comprises: contacting the adapter strands (1004) with polymerase and nucleotides; or contacting the adapter strands (1004) with pre-synthesized oligonucleotide strands under conditions that cause the pre-synthesized oligonucleotide strands to hybridize with the overhanging regions (1104) of the adapter strands (1004).

Clause 19. The method of any of clauses 15-18, wherein the substrate (102) comprises glass or silicon and functionalization (104) comprising silane groups or agarose.

Clause 20. The method of any of clauses 15-18, further comprising, washing the substrate (102) to remove the oligonucleotide strands (1002) without removing the single-stranded oligonucleotide linkers (110).

### CONCLUSION

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

The terms “a,” “an,” “the,” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced. As used herein, “approximately” or “about” or similar referents denote a range of ±10% of the stated value.

For ease of understanding, the process discussed in this disclosure is delineated as separate operations represented as independent blocks. However, these separately delineated operations should not be construed as necessarily order dependent in their performance. The order in which the processes described is not intended to be construed as a limitation, and unless other otherwise contradicted by context any number of the described process blocks may be combined in any order to implement the process or an alternate process. Moreover, it is also possible that one or more of the provided operations is modified or omitted.

Certain embodiments are described herein, including the best mode known to the inventors for carrying out the invention. Of course, variations on these described embodiments will become apparent to those of ordinary skill in the art upon reading the foregoing description. Skilled artisans will know how to employ such variations as appropriate, and the embodiments disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

Furthermore, references have been made to publications, patents and/or patent applications throughout this specification. Each of the cited references is individually incorporated herein by reference for its particular cited teachings as well as for all that it discloses.

1. A method for solid-phase synthesis of polymers, the method comprising:
generating polymer strands by adding monomers to free ends of linkers attached to a substrate;
contacting the linkers with a linker cleavage agent that cleaves the linkers at a recognition site thereby releasing the polymer strands from the substrate and generating truncated linkers; and
regenerating the linkers from the truncated linkers.

2. The method of claim 1, wherein the linkers comprise single-stranded oligonucleotide linkers and the linker cleavage agent comprises a restriction endonuclease that cleaves the linkers within the recognition site.

3. The method of claim 2, further comprising, prior to contacting the linkers with the linker cleavage agent, contacting the linkers with linker complement strands under conditions that cause the linker complement strands to hybridize with the linkers, wherein the recognition site is a double-stranded oligonucleotide sequence formed from hybridization of the linkers and the linker complement strands.

4. The method of claim 1, wherein the linkers comprise single-stranded oligonucleotide linkers and regenerating the linkers comprises:
contacting the truncated linkers with regeneration templates under conditions that cause the regeneration templates to hybridize with the truncated linkers; and
(i) extending the truncated linkers by addition of polymerase and nucleotides; or
(ii) contacting the regeneration templates with linker replacement strands and ligase.

5. The method of claim 1, wherein the linkers comprise polypeptide linkers and the linker cleavage agent comprises a protease that cleaves the linkers at the recognition site.

6. The method of claim 1, wherein the linkers comprise polypeptide linkers and the linker cleavage agent comprises a chemical cleavage agent.

7. The method of claim 1, wherein the linkers comprise polypeptide linkers and regenerating the linkers comprises performing solid-phase synthesis of polypeptides to extend the truncated linkers.

8. The method of claim 1, wherein the polymer strands comprise oligonucleotides and the method further comprises removing scars from the polymer strands by amplification with polymerase chain reaction (PCR).

9. A method for solid-phase synthesis of polymers, the method comprising:
a. generating polymer strands by adding monomers to free ends of linkers attached to a substrate;
b. contacting the linkers with a linker cleavage agent that cleaves at points of attachment between the linkers and the polymer strands; and
c. washing the substrate to remove the polymer strands without removing the linkers from the substrate.

10. The method of claim 9, further comprising repeating steps a-c at least once.

11. The method of claim 9, wherein the linkers comprise single-stranded oligonucleotide linkers and the method further comprises, prior to contacting the linkers with the linker cleavage agent, contacting the linkers with linker complement strands under conditions that cause the linker complement strands to hybridize with the linkers.

12. The method of claim 11, wherein the linker cleavage agent comprises a restriction endonuclease having a recognition site within a double-stranded oligonucleotide sequence formed by hybridization of the linkers with the linker complement strands and a cleavage site at the point of attachment of the linkers to the polymer strands.

13. The method of claim 11, wherein the linkers comprise deoxyribonucleic acid (DNA) strands having uracil bases at the free ends and the linker cleavage agent comprises Uracil DNA. Glycosylase (UDG).

14. The method of claim 9, wherein the linkers comprise poly peptide linkers and the linker cleavage agent comprises a protease that cleaves at the points of attachment between the polypeptide linkers and the polymer strands.

15. A method of solid-phase synthesis of oligonucleotides, the method comprising:
contacting single-stranded oligonucleotide linkers attached to a substrate with adapter strands under conditions that cause the adapter strands to hybridize with the single-stranded oligonucleotide linkers;
generating oligonucleotide strands that are (i) covalently attached to the adapter strands or (ii) partially hybridized to the adapter strands; and
releasing the oligonucleotide strands from the substrate by denaturing the single-stranded oligonucleotide linkers and the adapter strands.

16. The method of claim 15, wherein the single-stranded oligonucleotide linkers are oriented with 3′-ends attached to the substrate and free 5′-ends; and
generating the oligonucleotide strands that are (i) covalently attached to the adapter strands comprises extending the adapter strands by non-templated oligonucleotide synthesis thereby creating the oligonucleotide strands.

17. The method of claim 15, wherein the single-stranded oligonucleotide linkers are oriented with 5′-ends attached to the substrate and free 3′-ends, wherein the 3′-ends are modified to prevent extension, and generating the oligonucleotide strands (ii) partially hybridized to the adapter strands comprises:
generating anchoring oligonucleotide strand fragments complementary to overhanging regions of the adapter strands; and
extending the anchoring oligonucleotide strand fragments by non-templated oligonucleotide synthesis thereby creating the oligonucleotide strands.

18. The method of claim 17, wherein the generating the anchoring oligonucleotide stand fragments comprises:
contacting the adapter strands with polymerase and nucleotides; or
contacting the adapter strands with pre-synthesized oligonucleotide strands under conditions that cause the pre-synthesized oligonucleotide strands to hybridize with the overhanging regions of the adapter strands.

19. The method of claim 15, wherein the substrate comprises glass or silicon and functionalization comprising silane groups or agarose.

20. The method of claim 15, further comprising, washing the substrate to remove the oligonucleotide strands without removing the single-stranded oligonucleotide linkers.


SEQUENCE LISTING (2 sequences)

SEQ ID NO: 1 - protein, 9 aa, Artificial Sequence
  Recognition site for tobacco etch virus (TEV) protease
  Xaa (7)..(7): Is Gly or Ser
ENLYFQXAA

SEQ ID NO: 2 - protein, 5 aa, Artificial Sequence
  Recognition site of Enterokinase
DDDDK